SEQUENCE LISTING (340 sequences)

SEQ ID NO: 1 - protein, 34 aa, Momordica cochinchinensis
GGVCPKILQRCRRDSDCPGACICRGNGYCGSGSD

SEQ ID NO: 2 - protein, 34 aa, synthetic construct
GGBCPKILQRCRRDSDCPGACICRGAGYCGSGSD

SEQ ID NO: 3 - protein, 32 aa, synthetic construct
GGBCPKILQRCRRDSDCPGACICRGAGYCGSG

SEQ ID NO: 4 - protein, 32 aa, synthetic construct
GGVCPKILQRCRRDSDCPGACICRGNGYCGSG

SEQ ID NO: 5 - protein, 18 aa, synthetic construct
RGGRLCYCRRRFCVCVGR

SEQ ID NO: 6 - protein, 18 aa, synthetic construct
GGRLCYCRRRFCVCVGRR

SEQ ID NO: 7 - protein, 18 aa, synthetic construct
GGCLCYCRRRFCVCVCRR

SEQ ID NO: 8 - protein, 20 aa, synthetic construct
GGGRLCYCRRRFCVCVGRRG

SEQ ID NO: 9 - protein, 16 aa, synthetic construct
GRLCYCRRRFCVCVGR

SEQ ID NO: 10 - protein, 29 aa, Viola odorata
GLPVCGETCVGGTCNTPGCTCSWPVCTRN

SEQ ID NO: 11 - protein, 30 aa, Viola odorata
GIPCAESCVYIPCTVTALLGCSCSNRVCYN

SEQ ID NO: 12 - protein, 29 aa, Viola odorata
GLPVCGETCFGGTCNTPGCSCTWPICTRD

SEQ ID NO: 13 - protein, 37 aa, unidentified
GDPTFCGETCRVIPVCTYSAALGCTCDDRSDGLCKRN

SEQ ID NO: 14 - protein, 30 aa, Viola odorata
GIPCAESCVWIPCTVTALLGCSCSNKVCYN

SEQ ID NO: 15 - protein, 33 aa, Viola tricolor
GGTIFDCGESCFLGTCYTKGCSCGEWKLCYGTN

SEQ ID NO: 16 - protein, 30 aa, unidentified
GIPCGESCVWIPCISAALGCSCKNKVCYRN

SEQ ID NO: 17 - protein, 29 aa, synthetic construct
GLPVCGETCVGGTCNTPGCTCSWPVCTRN

SEQ ID NO: 18 - protein, 29 aa, synthetic construct
GLPVCGETCVGGTCNTPGCTCSWPVCTRN

SEQ ID NO: 19 - protein, 29 aa, synthetic construct
GLPVCGETCVGGTCNTPGCTCSWPVCTRN

SEQ ID NO: 20 - protein, 29 aa, synthetic construct
GLPVCGETCVGGTCNTPGCTCSWPVCTRN

SEQ ID NO: 21 - skipped

SEQ ID NO: 22 - protein, 28 aa, synthetic construct
LPVCGETCVGGTCNTPGCTCSWPVCTRN

SEQ ID NO: 23 - protein, 27 aa, synthetic construct
TCVGGTCNTPGCTCSWPVCTRNLPVCG

SEQ ID NO: 24 - protein, 29 aa, synthetic construct
GTCNTPGCTCSWPVCTRNGLPVCGETCVG

SEQ ID NO: 25 - protein, 27 aa, synthetic construct
GCTCSWPVCTRNGLPVCGETCVGGTCN

SEQ ID NO: 26 - protein, 28 aa, synthetic construct
CSWPVCTRNGLPVCGETCVGGTCNTPGC

SEQ ID NO: 27 - protein, 27 aa, synthetic construct
VCTRNGLPVCGETCVGGTCNTPGCTCS

SEQ ID NO: 28 - protein, 24 aa, synthetic construct
VCGETCVGGTCNTPGCTCSWPVCT

SEQ ID NO: 29 - protein, 29 aa, synthetic construct
RNGLPVCGETCVGGTCNTPGCTCSWPVCT

SEQ ID NO: 30 - protein, 30 aa, Viola odorata
GIPCGESCVWIPCISSAIGCSCKSKVCYRN

SEQ ID NO: 31 - protein, 24 aa, synthetic construct
VCGETCVGGTCNTPGCTCSWPVCT

SEQ ID NO: 32 - protein, 29 aa, synthetic construct
GLPVAGETCVGGTCNTPGATCSWPVCTRN

SEQ ID NO: 33 - protein, 30 aa, Viola odorata
GLPTCGETCFGGTCNTPGCSCSSWPICTRN

SEQ ID NO: 34 - protein, 30 aa, Viola odorata
GLPTCGETCFGGTCNTPGCTCDPWPICTRD

SEQ ID NO: 35 - protein, 29 aa, Viola odorata
GLPVCGETCTLGTCYTQGCTCSWPICKRN

SEQ ID NO: 36 - protein, 31 aa, Viola odorata
GTLPCGESCVWIPCISSVVGCSCKSKVCYKN

SEQ ID NO: 37 - protein, 31 aa, Viola odorata
GTLPCGESCVWIPCISAVVGCSCKSKVCYKN

SEQ ID NO: 38 - protein, 29 aa, Viola odorata
GLPVCGETCVGGTCNTPGCTCSWPVCTRD

SEQ ID NO: 39 - protein, 29 aa, Viola odorata
GAPICGESCFTGKCYTVQCSCSWPVCTRN

SEQ ID NO: 40 - protein, 31 aa, unidentified
SIPCGESCVFIPCTVTALLGCSCKSKVCYKN

SEQ ID NO: 41 - protein, 30 aa, Viola odorata
GIPCGESCVYIPCLTSAIGCSCKSKVCYRN

SEQ ID NO: 42 - protein, 30 aa, Viola odorata
GIPCGESCVWIPCLTSAVGCSCKSKVCYRN

SEQ ID NO: 43 - protein, 31 aa, Viola odorata
GSIPCAESCVYIPCFTGIAGCSCKNKVCYYN

SEQ ID NO: 44 - protein, 30 aa, Viola biflora
GIPCGESCVWIPCITSAIGCSCKSKVCYRN

SEQ ID NO: 45 - protein, 29 aa, Viola odorata
GLPVCGETCVGGTCNTPGCSCSWPVCTRN

SEQ ID NO: 46 - protein, 29 aa, Viola odorata
GLPICGETCVGGTCNTPGCSCSWPVCTRN

SEQ ID NO: 47 - protein, 29 aa, Viola odorata
GLPVCGETCTLGKCYTAGCSCSWPVCYRN

SEQ ID NO: 48 - protein, 31 aa, Viola odorata
GSIPCAESCVYIPCITGIAGCSCKNKVCYYN

SEQ ID NO: 49 - protein, 29 aa, Viola odorata
GLPVCGETCVGGTCNTPGCTCSWPVCTRN

SEQ ID NO: 50 - protein, 30 aa, Hybanthus parviflorus
GIPCAESCVYIPCTITALLGCSCKNKVCYN

SEQ ID NO: 51 - protein, 31 aa, unidentified
GVIPCGESCVFIPCISTLLGCSCKNKVCYRN

SEQ ID NO: 52 - protein, 30 aa, unidentified
GIPCGESCVFIPCITSVAGCSCKSKVCYRN

SEQ ID NO: 53 - protein, 30 aa, unidentified
KIPCGESCVWIPCVTSIFNCKCENKVCYHD

SEQ ID NO: 54 - protein, 30 aa, unidentified
KIPCGESCVWIPCLTSVFNCKCENKVCYHD

SEQ ID NO: 55 - protein, 29 aa, unidentified
AIPCGESCVWIPCISAAIGCSCKNKVCYR

SEQ ID NO: 56 - protein, 30 aa, Viola odorata
GIPCGESCVWIPCISSAIGCSCKNKVCYRN

SEQ ID NO: 57 - protein, 30 aa, Viola odorata
GIPCGESCVWIPCLTSAIGCSCKSKVCYRN

SEQ ID NO: 58 - protein, 30 aa, Viola odorata
GTPCGESCVWIPCISSAVGCSCKNKVCYKN

SEQ ID NO: 59 - protein, 31 aa, Viola odorata
GTLPCGESCVWIPCISAAVGCSCKSKVCYKN

SEQ ID NO: 60 - protein, 30 aa, Viola odorata
SIPCGESCVWIPCTITALAGCKCKSKVCYN

SEQ ID NO: 61 - protein, 30 aa, Viola odorata
GIPCGESCVYIPCLTSAVGCSCKSKVCYRN

SEQ ID NO: 62 - protein, 30 aa, Viola odorata
GTPCGESCVYIPCISGVIGCSCTDKVCYLN

SEQ ID NO: 63 - protein, 30 aa, synthetic construct
GLPVCGETCFGGTCNTPGCSCDPWPMCSRN

SEQ ID NO: 64 - protein, 29 aa, Viola arvensis
GVPICGETCVGGTCNTPGCSCSWPVCTRN

SEQ ID NO: 65 - protein, 29 aa, Viola arvensis
GLPICGETCVGGSCNTPGCSCSWPVCTRN

SEQ ID NO: 66 - protein, 29 aa, Viola arvensis
GVPICGETCTLGTCYTAGCSCSWPVCTRN

SEQ ID NO: 67 - protein, 30 aa, Viola arvensis
GVPVCGETCFGGTCNTPGCSCDPWPVCSRN

SEQ ID NO: 68 - protein, 30 aa, Viola arvensis
GLPVCGETCFGGTCNTPGCSCETWPVCSRN

SEQ ID NO: 69 - protein, 31 aa, unidentified
GVIPCGESCVFIPCISAAIGCSCKNKVCYRN

SEQ ID NO: 70 - protein, 28 aa, unidentified
GTACGESCYVLPCFTVGCTCTSSQCFKN

SEQ ID NO: 71 - protein, 30 aa, unidentified
GIPCGESCVFIPCLTTVAGCSCKNKVCYRN

SEQ ID NO: 72 - protein, 30 aa, unidentified
GFPCGESCVFIPCISAAIGCSCKNKVCYRN

SEQ ID NO: 73 - protein, 29 aa, Viola arvensis
  VARIANT 28: note = Any amino acid
GLPVCGETCVGGTCNTPGCSCSRPVCTXN

SEQ ID NO: 74 - protein, 31 aa, unidentified
SISCGESCAMISFCFTEVIGCSCKNKVCYLN

SEQ ID NO: 75 - protein, 27 aa, synthetic construct
ALETQKPNHLEEALVAFAKKGNLGGLP

SEQ ID NO: 76 - protein, 30 aa, unidentified
GIPCGESCHYIPCVTSAIGCSCRNRSCMRN

SEQ ID NO: 77 - protein, 30 aa, unidentified
GIPCGDSCHYIPCVTSTIGCSCTNGSCMRN

SEQ ID NO: 78 - protein, 20 aa, synthetic construct
GVKSSETTLMFLKEMQLKLP

SEQ ID NO: 79 - protein, 30 aa, unidentified
GLPVCGETCFTGTCYTNGCTCDPWPVCTRN

SEQ ID NO: 80 - protein, 30 aa, Viola odorata
GLPVCGETCFGGTCNTPGCICDPWPVCTRN

SEQ ID NO: 81 - protein, 29 aa, Viola odorata
SAIACGESCVYIPCFIPGCSCRNRVCYLN

SEQ ID NO: 82 - protein, 31 aa, unidentified
SISCGESCVYIPCTVTALVGCTCKDKVCYLN

SEQ ID NO: 83 - protein, 32 aa, unidentified
GSPIQCAETCFIGKCYTEELGCTCTAFLCMKN

SEQ ID NO: 84 - protein, 32 aa, unidentified
GSPRQCAETCFIGKCYTEELGCTCTAFLCMKN

SEQ ID NO: 85 - protein, 31 aa, unidentified
GSVPCGESCVYIPCFTGIAGCSCKSKVCYYN

SEQ ID NO: 86 - protein, 29 aa, unidentified
GEIPCGESCVYLPCFLPNCYCRNHVCYLN

SEQ ID NO: 87 - protein, 30 aa, unidentified
SISCGETCTTFNCWIPNCKCNHHDKVCYWN

SEQ ID NO: 88 - protein, 26 aa, synthetic construct
CAETCVVLPCFIVPGCSCKSSVCYFN

SEQ ID NO: 89 - protein, 27 aa, synthetic construct
CAETCIYIPCFTEAVGCKCKDKVCYKN

SEQ ID NO: 90 - protein, 30 aa, unidentified
GIPCGESCVFIPCISGVIGCSCKSKVCYRN

SEQ ID NO: 91 - protein, 28 aa, unidentified
GIACGESCAYFGCWIPGCSCRNKVCYFN

SEQ ID NO: 92 - protein, 30 aa, unidentified
GTPCGESCVYIPCFTAVVGCTCKDKVCYLN

SEQ ID NO: 93 - protein, 30 aa, unidentified
GTPCAESCVYLPCFTGVIGCTCKDKVCYLN

SEQ ID NO: 94 - protein, 29 aa, unidentified
GNIPCGESCIFFPCFNPGCSCKDNLCYYN

SEQ ID NO: 95 - protein, 27 aa, synthetic construct
CGETCVILPCISAALGCSCKDTVCYKN

SEQ ID NO: 96 - protein, 27 aa, synthetic construct
CGETCVIFPCISAAFGCSCKDTVCYKN

SEQ ID NO: 97 - protein, 31 aa, unidentified
GSVPCGESCVWIPCISGIAGCSCKNKVCYLN

SEQ ID NO: 98 - protein, 27 aa, synthetic construct
CGETCLFIPCIFSVVGCSCSSKVCYRN

SEQ ID NO: 99 - protein, 25 aa, synthetic construct
CGETCVTGTCYTPGCACDWPVCKRD

SEQ ID NO: 100 - protein, 27 aa, synthetic construct
CGETCIWGRCYSENIGCHCGFGICTLN

SEQ ID NO: 101 - protein, 27 aa, synthetic construct
CGETCLFIPCLTSVFGCSCKNRGCYKI

SEQ ID NO: 102 - protein, 26 aa, synthetic construct
CGETCVVDTRCYTKKCSCAWPVCMRN

SEQ ID NO: 103 - protein, 23 aa, synthetic construct
CVWIPCISAAIGCSCKSKVCYRN

SEQ ID NO: 104 - protein, 28 aa, synthetic construct
CGESCVYIPCTVTALLGCSCKDKVCYKN

SEQ ID NO: 105 - protein, 26 aa, synthetic construct
CGETCKVTKRCSGQGCSCLKGRSCYD

SEQ ID NO: 106 - protein, 26 aa, synthetic construct
CGETCVVLPCFIVPGCSCKSSVCYFN

SEQ ID NO: 107 - protein, 27 aa, synthetic construct
CGETCIYIPCFTEAVGCKCKDKVCYKN

SEQ ID NO: 108 - protein, 33 aa, Viola tricolor
GGTIFDCGESCFLGTCYTKGCSCGEWKLCYGEN

SEQ ID NO: 109 - protein, 31 aa, unidentified
GSVLNCGETCLLGTCYTTGCTCNKYRVCTKD

SEQ ID NO: 110 - protein, 30 aa, Viola odorata
GIPCAESCVWIPCTVTALLGCSCSNNVCYN

SEQ ID NO: 111 - protein, 30 aa, Viola odorata
GIPCGESCVWIPCISAAIGCSCKSKVCYRN

SEQ ID NO: 112 - protein, 27 aa, Viola odorata
SAISCGETCFKFKCYTPRCSCSYPVCK

SEQ ID NO: 113 - protein, 31 aa, Viola odorata
GSIPACGESCFKGKCYTPGCSCSKYPLCAKN

SEQ ID NO: 114 - protein, 29 aa, Viola odorata
GLVPCGETCFTGKCYTPGCSCSYPICKKN

SEQ ID NO: 115 - protein, 29 aa, Viola odorata
GLPCGETCFTGKCYTPGCSCSYPICKKIN

SEQ ID NO: 116 - protein, 30 aa, Viola odorata
GIPCGESCVWIPCISAAIGCSCKNKVCYRN

SEQ ID NO: 117 - protein, 30 aa, Viola odorata
GIPCGESCVYIPCTVTALAGCKCKSKVCYN

SEQ ID NO: 118 - protein, 31 aa, Viola odorata
GTLPCGESCVWIPCISSVVGCSCKSKVCYKD

SEQ ID NO: 119 - protein, 30 aa, Viola odorata
GIPCGESCVWIPCLTSAIGCSCKSKVCYRD

SEQ ID NO: 120 - protein, 29 aa, Viola odorata
GLPVCGETCVTGSCYTPGCTCSWPVCTRN

SEQ ID NO: 121 - protein, 29 aa, Viola odorata
GLPICGETCVGGTCNTPGCTCSWPVCTRN

SEQ ID NO: 122 - protein, 31 aa, Viola odorata
GLPTCGETCFGGTCNTPGCTCDSSWPICTHN

SEQ ID NO: 123 - protein, 30 aa, Viola odorata
GLPTCGETCFGGTCNTPGCTCDPWPVCTHN

SEQ ID NO: 124 - protein, 31 aa, Viola odorata
DIFCGETCAFIPCITHVPGTCSCKSKVCYFN

SEQ ID NO: 125 - protein, 29 aa, synthetic construct
GLPVCGETCVGGTCNTPGCTCSWDKCTRN

SEQ ID NO: 126 - protein, 29 aa, synthetic construct
GLPVCGETCVGGTCNTPGCTCSKNKCTRN

SEQ ID NO: 127 - protein, 30 aa, synthetic construct
  VARIANT 6: note = Any amino acid
GIPCGXSCVWIPCISSAIGCSCKSKVCYRN

SEQ ID NO: 128 - protein, 30 aa, synthetic construct
  VARIANT 23: note = Any amino acid
  VARIANT 25: note = Any amino acid
GIPCGESCVWIPCISSAIGCSCXSXVCYRN

SEQ ID NO: 129 - protein, 30 aa, synthetic construct
  VARIANT 29: note = Any amino acid
GIPCGESCVWIPCISSAIGCSCKSKVCYXN

SEQ ID NO: 130 - protein, 30 aa, synthetic construct
  VARIANT 23: note = Any amino acid
  VARIANT 25: note = Any amino acid
  VARIANT 29: note = Any amino acid
GIPCGESCVWIPCISSAIGCSCXSXVCYXN

SEQ ID NO: 131 - protein, 29 aa, Viola odorata
GLPVCGETCVGGTCNTPGCTCSWPVCTRN

SEQ ID NO: 132 - protein, 29 aa, Viola odorata
GLPVCGETCVGGTCNTPGCTCSWPVCTRN

SEQ ID NO: 133 - protein, 29 aa, Viola odorata
GLPVCGETCFGGTCNTPGCSCTWPICTRD

SEQ ID NO: 134 - protein, 29 aa, Viola odorata
GLPVCGETCFGGTCNTPGCSCTWPICTRD

SEQ ID NO: 135 - protein, 31 aa, unidentified
GSVFNCGETCVLGTCYTPGCTCNTYRVCTKD

SEQ ID NO: 136 - protein, 30 aa, unidentified
GLPTCGETCFGGTCNTPGCSCSSWPICTRD

SEQ ID NO: 137 - protein, 30 aa, unidentified; identical to SEQ ID NO: 136

SEQ ID NO: 138 - protein, 29 aa, unidentified
GLPVCGETCFGGTCNTPGCSCTDPICTRD

SEQ ID NO: 139 - protein, 28 aa, unidentified
GSLCGDTCFVLGCNDSSCSCNYPICVKD

SEQ ID NO: 140 - protein, 30 aa, unidentified
GLPVCGETCFGGTCNTPGCACDPWPVCTRD

SEQ ID NO: 141 - protein, 30 aa, unidentified
GLPVCGESCFGGTCNTPGCACDPWPVCTRD

SEQ ID NO: 142 - protein, 29 aa, unidentified
GLPVCGESCFGGSCYTPGCSCTWPICTRD

SEQ ID NO: 143 - protein, 30 aa, unidentified
GIPCAESCVYIPCTITALLGCKCQDKVCYD

SEQ ID NO: 144 - protein, 30 aa, unidentified
GIPCAESCVYIPCTITALLGCKCKDQVCYN

SEQ ID NO: 145 - protein, 28 aa, synthetic construct
CGETCVGGTCNTPGCTCSWPVCRRKRRR

SEQ ID NO: 146 - protein, 31 aa, synthetic construct
CGETCRRKRRRCNTPGCTCSWPVCTRNGLPV

SEQ ID NO: 147 - protein, 34 aa, synthetic construct
CGETCVGGTCNTRRKRRRGCTCSWPVCTRNGLPV

SEQ ID NO: 148 - protein, 32 aa, synthetic construct
CGETCVGGTCNTPGCTCRRKRRRVCTRNGLPV

SEQ ID NO: 149 - protein, 31 aa, synthetic construct
CGETCVGGTCNTPGCTCRRKRRRCTRNGLPV

SEQ ID NO: 150 - protein, 31 aa, synthetic construct
CGETCVGGTCRRKRRRCTCSWPVCTRNGLPV

SEQ ID NO: 151 - protein, 30 aa, unidentified
GVPCAESCVYIPCISTVLGCSCSNQVCYRN

SEQ ID NO: 152 - protein, 31 aa, unidentified
GFIPCGETCIWDKTCHAAGCSCSVANICVRN

SEQ ID NO: 153 - protein, 34 aa, unidentified
GADGFCGESCYVIPCISYLVGCSCDTIEKVCKRN

SEQ ID NO: 154 - protein, 33 aa, Viola odorata
GGTIFDCGETCFLGTCYTPGCSCGNYGFCYGTN

SEQ ID NO: 155 - protein, 33 aa, Viola odorata
GGTIFDCGESCFLGTCYTAGCSCGNWGLCYGTN

SEQ ID NO: 156 - protein, 33 aa, Viola odorata
GGTIFDCGETCFLGTCYTAGCSCGNWGLCYGTN

SEQ ID NO: 157 - protein, 30 aa, Viola odorata
GVPCGESCVFIPCITGVIGCSCSSNVCYLN

SEQ ID NO: 158 - protein, 30 aa, Viola odorata
GIPCAESCVWIPCTVTALVGCSCSDKVCYN

SEQ ID NO: 159 - protein, 29 aa, Viola biflora
GLPVCGETCFGGTCNTPGCSCSYPICTRN

SEQ ID NO: 160 - protein, 29 aa, Viola biflora
GLPVCGETCFGGTCNTPGCTCSYPICTRN

SEQ ID NO: 161 - protein, 29 aa, Viola biflora
GLPVCGETCAFGSCYTPGCSCSWPVCTRN

SEQ ID NO: 162 - protein, 29 aa, Viola biflora
GLPVCGETCFGGRCNTPGCTCSYPICTRN

SEQ ID NO: 163 - protein, 30 aa, Viola biflora
GIPCAESCVWIPCTVTALIGCGCSNKVCYN

SEQ ID NO: 164 - protein, 31 aa, Viola biflora
GTIPCGESCVFIPCLTSALGCSCKSKVCYKN

SEQ ID NO: 165 - protein, 31 aa, Viola biflora
GTFPCGESCVFIPCLTSAIGCSCKSKVCYKN

SEQ ID NO: 166 - protein, 31 aa, Viola biflora
GLLPCAESCVYIPCLTTVIGCSCKSKVCYKN

SEQ ID NO: 167 - protein, 30 aa, Viola biflora
GIPCGESCVWIPCLTSTVGCSCKSKVCYRN

SEQ ID NO: 168 - protein, 31 aa, Viola biflora
GTFPCGESCVWIPCISKVIGCACKSKVCYKN

SEQ ID NO: 169 - protein, 30 aa, Viola biflora
GIPCGESCVWIPCLTSAVGCPCKSKVCYRN

SEQ ID NO: 170 - protein, 30 aa, unidentified
GIPCGESCVYLPCFTAPLGCSCSSKVCYRN

SEQ ID NO: 171 - protein, 30 aa, unidentified
GIPCGESCVWIPCLTATIGCSCKSKVCYRN

SEQ ID NO: 172 - protein, 29 aa, unidentified
GIPCAESCVYLPCVTIVIGCSCKDKVCYN

SEQ ID NO: 173 - protein, 30 aa, unidentified
GIPCAESCVWIPCTVTALLGCSCKDKVCYN

SEQ ID NO: 174 - protein, 34 aa, unidentified
GRLCGERCVIERTRAWCRTVGCICSLHTLECVRN

SEQ ID NO: 175 - protein, 29 aa, unidentified
GLPVCGETCVGGTCNTPGCGCSWPVCTRN

SEQ ID NO: 176 - protein, 29 aa, unidentified
GLPVCGETCVGGTCNTPGCACSWPVCTRN

SEQ ID NO: 177 - protein, 31 aa, unidentified
GSIPCGESCVYIPCISSLLGCSCKSKVCYKN

SEQ ID NO: 178 - protein, 30 aa, unidentified
GIPCAESCVYIPCLTSAIGCSCKSKVCYRN

SEQ ID NO: 179 - protein, 29 aa, unidentified
GIPCGESCVYLPCFTTIIGCKCQGKVCYH

SEQ ID NO: 180 - protein, 31 aa, unidentified
GSIPCGESCVFIPCISSVVGCSCKNKVCYKN

SEQ ID NO: 181 - protein, 31 aa, unidentified
GTIPCGESCVFIPCLTSAIGCSCKSKVCYKN

SEQ ID NO: 182 - protein, 31 aa, unidentified
GSIPCGESCVYIPCISSLLGCSCESKVCYKN

SEQ ID NO: 183 - protein, 31 aa, unidentified
GSIPCGESCVFIPCISSIVGCSCKSKVCYKN

SEQ ID NO: 184 - protein, 30 aa, unidentified
GIPCGESCVFIPCLTSAIGCSCKSKVCYRN

SEQ ID NO: 185 - protein, 30 aa, unidentified
GVPCGESCVWIPCLTSIVGCSCKNNVCTLN

SEQ ID NO: 186 - protein, 31 aa, unidentified
GVIPCGESCVFIPCISSVLGCSCKNKVCYRN

SEQ ID NO: 187 - protein, 29 aa, unidentified
GHPTCGETCLLGTCYTPGCTCKRPVCYKN

SEQ ID NO: 188 - protein, 30 aa, unidentified
GSAILCGESCTLGECYTPGCTCSWPICTKN

SEQ ID NO: 189 - protein, 29 aa, unidentified
GHPICGETCVGNKCYTPGCTCTWPVCYRN

SEQ ID NO: 190 - protein, 31 aa, unidentified
GSIPCGEGCVFIPCISSIVGCSCKSKVCYKN

SEQ ID NO: 191 - protein, 30 aa, unidentified
GIPCGEGCVYLPCFTAPLGCSCSSKVCYRN

SEQ ID NO: 192 - protein, 30 aa, unidentified
GIPCGESCVWIPCLTAAIGCSCSSKVCYRN

SEQ ID NO: 193 - protein, 30 aa, unidentified
GVPCGESCVWIPCLTSAIGCSCKSSVCYRN

SEQ ID NO: 194 - protein, 30 aa, unidentified
GIPCGESCVLIPCISSVIGCSCKSKVCYRN

SEQ ID NO: 195 - protein, 31 aa, unidentified
GVIPCGESCVFIPCISSVIGCSCKSKVCYRN

SEQ ID NO: 196 - protein, 30 aa, unidentified
GAGCIETCYTFPCISEMINCSCKNSRCQKN

SEQ ID NO: 197 - protein, 30 aa, unidentified
GIPCGESCVWIPCISSAIGCSCKNKVCYRK

SEQ ID NO: 198 - protein, 30 aa, unidentified
GIPCGESCVWIPCISGAIGCSCKSKVCYRN

SEQ ID NO: 199 - protein, 30 aa, unidentified
TIPCAESCVWIPCTVTALLGCSCKDKVCYN

SEQ ID NO: 200 - protein, 29 aa, unidentified
GLPICGETCTLGTCYTVGCTCSWPICTRN

SEQ ID NO: 201 - protein, 29 aa, synthetic construct
ALPVCGETCVGGTCNTPGCTCSWPVCTRN

SEQ ID NO: 202 - protein, 29 aa, synthetic construct
GAPVCGETCVGGTCNTPGCTCSWPVCTRN

SEQ ID NO: 203 - protein, 29 aa, synthetic construct
GLAVCGETCVGGTCNTPGCTCSWPVCTRN

SEQ ID NO: 204 - protein, 29 aa, synthetic construct
GLPACGETCVGGTCNTPGCTCSWPVCTRN

SEQ ID NO: 205 - protein, 29 aa, synthetic construct
GLPVCAETCVGGTCNTPGCTCSWPVCTRN

SEQ ID NO: 206 - protein, 29 aa, synthetic construct
GLPVCGATCVGGTCNTPGCTCSWPVCTRN

SEQ ID NO: 207 - protein, 29 aa, synthetic construct
GLPVCGEACVGGTCNTPGCTCSWPVCTRN

SEQ ID NO: 208 - protein, 29 aa, synthetic construct
GLPVCGETCAGGTCNTPGCTCSWPVCTRN

SEQ ID NO: 209 - protein, 29 aa, synthetic construct
GLPVCGETCVAGTCNTPGCTCSWPVCTRN

SEQ ID NO: 210 - protein, 29 aa, synthetic construct
GLPVCGETCVGATCNTPGCTCSWPVCTRN

SEQ ID NO: 211 - protein, 29 aa, synthetic construct
GLPVCGETCVGGACNTPGCTCSWPVCTRN

SEQ ID NO: 212 - protein, 29 aa, synthetic construct
GLPVCGETCVGGTCATPGCTCSWPVCTRN

SEQ ID NO: 213 - protein, 29 aa, synthetic construct
GLPVCGETCVGGTCNAPGCTCSWPVCTRN

SEQ ID NO: 214 - protein, 29 aa, synthetic construct
GLPVCGETCVGGTCNTAGCTCSWPVCTRN

SEQ ID NO: 215 - protein, 29 aa, synthetic construct
GLPVCGETCVGGTCNTPACTCSWPVCTRN

SEQ ID NO: 216 - protein, 29 aa, synthetic construct
GLPVCGETCVGGTCNTPGCACSWPVCTRN

SEQ ID NO: 217 - protein, 29 aa, synthetic construct
GLPVCGETCVGGTCNTPGCTCAWPVCTRN

SEQ ID NO: 218 - protein, 29 aa, synthetic construct
GLPVCGETCVGGTCNTPGCTCSAPVCTRN

SEQ ID NO: 219 - protein, 29 aa, synthetic construct
GLPVCGETCVGGTCNTPGCTCSWAVCTRN

SEQ ID NO: 220 - protein, 29 aa, synthetic construct
GLPVCGETCVGGTCNTPGCTCSWPACTRN

SEQ ID NO: 221 - protein, 29 aa, synthetic construct
GLPVCGETCVGGTCNTPGCTCSWPVCARN

SEQ ID NO: 222 - protein, 29 aa, synthetic construct
GLPVCGETCVGGTCNTPGCTCSWPVCTAN

SEQ ID NO: 223 - protein, 29 aa, synthetic construct
GLPVCGETCVGGTCNTPGCTCSWPVCTRA

SEQ ID NO: 224 - protein, 31 aa, Clitoria ternatea
GVIPCGESCVFIPCISTVIGCSCKNKVCYRN

SEQ ID NO: 225 - protein, 31 aa, Clitoria ternatea
GVPCAESCVWIPCTVTALLGCSCKDKVCYLN

SEQ ID NO: 226 - protein, 29 aa, unidentified
GIPCGESCHIPCVTSAIGCSCRNRSCMRN

SEQ ID NO: 227 - protein, 31 aa, Viola pinetorum
GSQSCGESCVLIPCISGVIGCSCSSMICYFN

SEQ ID NO: 228 - protein, 30 aa, Viola pinetorum
GIPCGESCVFIPCLTAAIGCSCRSKVCYRN

SEQ ID NO: 229 - protein, 29 aa, Viola odorata
GLPVCGETCVGGTCNTPGCSCSIPVCTRN

SEQ ID NO: 230 - protein, 29 aa, Viola odorata
GLPVCGETCVGGTCNTPGCSCSWPVCFRD

SEQ ID NO: 231 - protein, 30 aa, Viola odorata
GAPVCGETCFGGTCNTPGCTCDPWPVCTND

SEQ ID NO: 232 - protein, 29 aa, Viola odorata
GLPVCGETCVGGTCNTPYCTCSWPVCTRD

SEQ ID NO: 233 - protein, 29 aa, Viola odorata
GLPVCGETCVGGTCNTEYCTCSWPVCTRD

SEQ ID NO: 234 - protein, 29 aa, Viola odorata
GLPVCGETCVGGTCNTPYCFCSWPVCTRD

SEQ ID NO: 235 - protein, 30 aa, Viola odorata
GIPCGESCVWIPCISGAIGCSCKSKVCYKN

SEQ ID NO: 236 - protein, 30 aa, Viola odorata
GIPCGESCVWIPCISSAIGCSCKNKVCFKN

SEQ ID NO: 237 - protein, 31 aa, Viola odorata
GSIPACGESCFRGKCYTPGCSCSKYPLCAKD

SEQ ID NO: 238 - protein, 31 aa, Viola odorata
GSIPACGESCFKGWCYTPGCSCSKYPLCAKD

SEQ ID NO: 239 - protein, 31 aa, unidentified
GSFPCGESCVFIPCISAIAGCSCKNKVCYKN

SEQ ID NO: 240 - protein, 30 aa, unidentified
GIPCGESCVFIPCITAAIGCSCKTKVCYRN

SEQ ID NO: 241 - protein, 31 aa, unidentified
GVIPCGESCVFIPCISAVLGCSCKSKVCYRN

SEQ ID NO: 242 - protein, 30 aa, unidentified
GIPCGETCVFMPCISGPMGCSCKHMVCYRN

SEQ ID NO: 243 - protein, 31 aa, unidentified; identical to SEQ ID NO: 186

SEQ ID NO: 244 - protein, 30 aa, unidentified
GSAFGCGETCVKGKCNTPGCVCSWPVCKKN

SEQ ID NO: 245 - protein, 29 aa, unidentified
APCGESCVFIPCISAVLGCSCKSKVCYRN

SEQ ID NO: 246 - protein, 31 aa, unidentified
GVPCGESCVWVPCTVTALMGCSCVREVCRKD

SEQ ID NO: 247 - protein, 30 aa, unidentified
GIPCAESCVWIPCTVTKMLGCSCKDKVCYN

SEQ ID NO: 248 - protein, 32 aa, unidentified
GGSIPCIETCVWTGCFLVPGCSCKSDKKCYLN

SEQ ID NO: 249 - protein, 32 aa, unidentified
GGSVPCIETCVWTGCFLVPGCSCKSDKKCYLN

SEQ ID NO: 250 - protein, 31 aa, unidentified
GDIPLCGETCFEGGNCRIPGCTCVWPFCSKN

SEQ ID NO: 251 - protein, 30 aa, Viola odorata
GLPTCGETCFGGTCNTPGCTCDPFPVCTHD

SEQ ID NO: 252 - protein, 29 aa, unidentified
GIPVCGETCVGGTCNTPGCSCSWPVCTRN

SEQ ID NO: 253 - protein, 29 aa, unidentified
GLPICGETCVGGTCNTPGCSCSWPVCTRN

SEQ ID NO: 254 - protein, 28 aa, unidentified
GIACGESCVFLGCFIPGCSCKSKVCYFN

SEQ ID NO: 255 - protein, 28 aa, unidentified
GIPCGETCVAFGCWIPGCSCKDKLCYYD

SEQ ID NO: 256 - protein, 25 aa, unidentified
KLCGETCFKFKCYTPGCSCSYFPCK

SEQ ID NO: 257 - protein, 31 aa, unidentified
GIPCGESCVFIPCTVTALLGCSCQNKVCYRD

SEQ ID NO: 258 - protein, 31 aa, unidentified
GVIPCGESCVFIPCISSVLGCSCKNKVCYRD

SEQ ID NO: 259 - protein, 31 aa, unidentified
GVIPCGESCVFIPCITAAVGCSCKNKVCYRD

SEQ ID NO: 260 - protein, 29 aa, unidentified
GLPVCGETCAGGTCNTPGCSCSWPICTRN

SEQ ID NO: 261 - protein, 29 aa, unidentified
GLPVCGETCAGGTCNTPGCSCTWPICTRN

SEQ ID NO: 262 - protein, 29 aa, unidentified
GLPVCGETCAGGRCNTPGCSCSWPVCTRN

SEQ ID NO: 263 - protein, 30 aa, unidentified
GLPVCGETCFGGTCNTPGCTCDPWPVCTRN

SEQ ID NO: 264 - protein, 30 aa, unidentified
GLPVCGETCFGGTCNTPGCSCDPWPVCTRN

SEQ ID NO: 265 - protein, 30 aa, unidentified
GLPICGETCFGGTCNTPGCICDPWPVCTRD

SEQ ID NO: 266 - protein, 29 aa, unidentified
GSHCGETCFFFGCYKPGCSCDELRQCYKN

SEQ ID NO: 267 - protein, 30 aa, unidentified
GVPCGESCVFIPCLTAVVGCSCSNKVCYLN

SEQ ID NO: 268 - protein, 30 aa, unidentified
GLPVCGETCFGGTCNTPGCACDPWPVCTRN

SEQ ID NO: 269 - protein, 31 aa, Clitoria ternatea
GVPCAESCVWIPCTVTALLGCSCKDKVCYLD

SEQ ID NO: 270 - protein, 31 aa, Clitoria ternatea
GIPCAESCVWIPCTVTALLGCSCKDKVCYLN

SEQ ID NO: 271 - protein, 31 aa, Clitoria ternatea
GIPCAESCVWIPCTVTALLGCSCKDKVCYLD

SEQ ID NO: 272 - protein, 30 aa, Clitoria ternatea
GIPCGESCVFIPCISSVVGCSCKSKVCYLD

SEQ ID NO: 273 - protein, 30 aa, Clitoria ternatea
GLPCGESCVFIPCITTVVGCSCKNKVCYNN

SEQ ID NO: 274 - protein, 30 aa, Clitoria ternatea
GLPCGESCVFIPCITTVVGCSCKNKVCYND

SEQ ID NO: 275 - protein, 31 aa, Clitoria ternatea
GTVPCGESCVFIPCITGIAGCSCKNKVCYIN

SEQ ID NO: 276 - protein, 31 aa, Clitoria ternatea
GTVPCGESCVFIPCITGIAGCSCKNKVCYID

SEQ ID NO: 277 - protein, 29 aa, Clitoria ternatea
HEPCGESCVFIPCITTVVGCSCKNKVCYN

SEQ ID NO: 278 - protein, 29 aa, Clitoria ternatea
HEPCGESCVFIPCITTVVGCSCKNKVCYD

SEQ ID NO: 279 - protein, 29 aa, Clitoria ternatea
GLPTCGETCTLGTCYVPDCSCSWPICMKN

SEQ ID NO: 280 - protein, 29 aa, Clitoria ternatea
GSAFCGETCVLGTCYTPDCSCTALVCLKN

SEQ ID NO: 281 - protein, 30 aa, Clitoria ternatea
GIPCGESCVFIPCITGIAGCSCKSKVCYRN

SEQ ID NO: 282 - protein, 30 aa, Clitoria ternatea
GIPCGESCVFIPCITAAIGCSCKSKVCYRN

SEQ ID NO: 283 - protein, 30 aa, Clitoria ternatea
GIPCGESCVFIPCISTVIGCSCKNKVCYRN

SEQ ID NO: 284 - protein, 31 aa, Clitoria ternatea
GIPCGESCVFIPCTVTALLGCSCKDKVCYKN

SEQ ID NO: 285 - protein, 29 aa, Viola tricolor
GVPICGESCVGGTCNTPGCSCSWPVCTTN

SEQ ID NO: 286 - protein, 29 aa, Viola tricolor
GLPICGETCVGGTCNTPGCFCTWPVCTRN

SEQ ID NO: 287 - protein, 29 aa, Viola tricolor
GLPVCGETCFTGSCYTPGCSCNWPVCNRN

SEQ ID NO: 288 - protein, 29 aa, Viola tricolor
GLPVCGETCVGGTCNTPGCSCSWPVCFRN

SEQ ID NO: 289 - protein, 31 aa, Viola tricolor
GLTPCGESCVWIPCISSVVGCACKSKVCYKD

SEQ ID NO: 290 - protein, 30 aa, unidentified
GTRCGETCFVLPCWSAKFGCYCQKGFCYRN

SEQ ID NO: 291 - protein, 26 aa, synthetic construct
  VARIANT 1: note = G, R, or L
  VARIANT 2: note = G, R, or L
  VARIANT 3: note = G, R, or L
  VARIANT 4: note = G, R, or L
  VARIANT 5: note = G, R, or L
  VARIANT 22: note = G, R, or L
  VARIANT 23: note = G, R, or L
  VARIANT 24: note = G, R, or L
  VARIANT 25: note = G, R, or L
  VARIANT 26: note = G, R, or L
  VARIANT 1..5: note = This region may encompass 0-5 residues
  VARIANT 22..26: note = This region may encompass 0-5 residues
XXXXXGRLCYCRRRFCVCVGRXXXXX

SEQ ID NO: 292 - protein, 17 aa, synthetic construct
GGRLCYCRRRFVCVGRR

SEQ ID NO: 293 - protein, 51 aa, synthetic construct
GGVCPKILQRCRRDSDCPGACICRGNGYCGSGSGGRLCYCRRRFCVCVGRR

SEQ ID NO: 294 - protein, 51 aa, synthetic construct
GGVCPKILQRCRRDSDCPGACICRGNGYCGSGSGGCLCYCRRRFCVCVCRR

SEQ ID NO: 295 - protein, 54 aa, synthetic construct
GGVCPKILQRCRRDSDCPGACICRGNGYCGSGSGGGRLCYCRRRFCVCVCGRRG

SEQ ID NO: 296 - protein, 49 aa, synthetic construct
GGVCPKILQRCRRDSDCPGACICRGNGYCGSGSGRLCYCRRRFCVCVGR

SEQ ID NO: 297 - protein, 53 aa, synthetic construct
  DISULFID 4..21: note = Intrachain disulfide bond
  DISULFID 11..23: note = Intrachain disulfide bond
  DISULFID 17..29: note = Intrachain disulfide bond
  VARIANT 34..53: note = This region may encompass one of the following sequences: GGRLCYCRRRFCVCVGRR, GGCLCYCRRRFCVCVCRR, GGGRLCYCRRRFCVCVGRRG or GRLCYCRRRFCVCVGR (SEQ ID NOS 299-302)
GGVCPKILQRCRRDSDCPGACICRGNGYCGSGSXXXXXXXXXXXXXXXXXXXX

SEQ ID NO: 298 - protein, 18 aa, Sus scrofa
  DISULFID 6..15: note = Intrachain disulfide bond
  DISULFID 8..13: note = Intrachain disulfide bond
  SITE 18: note = Amidated residue
RGGRLCYCRRRFCVCVGR

SEQ ID NO: 299 - protein, 18 aa, synthetic construct
  DISULFID 5..14: note = Intrachain disulfide bond
  DISULFID 7..12: note = Intrachain disulfide bond
GGRLCYCRRRFCVCVGRR

SEQ ID NO: 300 - protein, 18 aa, synthetic construct
  DISULFID 3..16: note = Intrachain disulfide bond
  DISULFID 5..14: note = Intrachain disulfide bond
  DISULFID 7..12: note = Intrachain disulfide bond
GGCLCYCRRRFCVCVCRR

SEQ ID NO: 301 - protein, 20 aa, synthetic construct
  DISULFID 6..15: note = Intrachain disulfide bond
  DISULFID 8..13: note = Intrachain disulfide bond
GGGRLCYCRRRFCVCVGRRG

SEQ ID NO: 302 - protein, 16 aa, synthetic construct
  DISULFID 4..13: note = Intrachain disulfide bond
  DISULFID 6..11: note = Intrachain disulfide bond
GRLCYCRRRFCVCVGR

SEQ ID NO: 303 - protein, 34 aa, Momordica cochinchinensis; identical to SEQ ID NO: 1

SEQ ID NO: 304 - protein, 34 aa, unidentified
GGVCPKILKKCRRDSDCPGACICRGNGYCGSGSD

SEQ ID NO: 305 - protein, 34 aa, unidentified
GGACPRILKKCRRDSDCPGACVCQGNGYCGSGSD

SEQ ID NO: 306 - protein, 34 aa, Momordica cochinchinensis
GGACPRILKKCRRDSDCPGACVCKGNGYCGSGSD

SEQ ID NO: 307 - protein, 34 aa, Momordica cochinchinensis
GGVCPKILQRCRRDSDCPGACICLGNGYCGSGSD

SEQ ID NO: 308 - protein, 32 aa, Momordica cochinchinensis
GGICPKILQRCRRDSDCPGACICRGNGYCGSD

SEQ ID NO: 309 - protein, 34 aa, Momordica cochinchinensis
GGVCPKILKKCRRDSDCPGACICRGNGYCSSGSD

SEQ ID NO: 310 - protein, 34 aa, Momordica cochinchinensis
GGVCPKILKKCRHDSDCPGACICRGNEYCGSGSD

SEQ ID NO: 311 - protein, 34 aa, Momordica cochinchinensis
GGACPRILKKCRRDSDCPGACICRGNGYCGSGSD

SEQ ID NO: 312 - protein, 34 aa, unidentified
GGACPKILQRCRRDSDCPSACICRGNGYCGSGSD

SEQ ID NO: 313 - protein, 34 aa, unidentified
GGACPKILQKCRRDSDCPGACVCQGNGYCGSGSD

SEQ ID NO: 314 - protein, 34 aa, unidentified
GGACPRILKQCRRDSDCPGACVCQGNGYCGSGSD

SEQ ID NO: 315 - protein, 34 aa, unidentified
GGACPRILKQCRRDSDCPGACICQGNGYCGSGSD

SEQ ID NO: 316 - protein, 34 aa, unidentified
GGACPRILKKCRRDSDCPGACVCRGNGYCGSGSD

SEQ ID NO: 317 - protein, 34 aa, unidentified
GGICPKILQRCRRDSDCPGACICRGNGYCGSGSD

SEQ ID NO: 318 - protein, 34 aa, unidentified
GGVCPKILKKCRHDSDCPGACICRGNGYCGSGSD

SEQ ID NO: 319 - protein, 34 aa, unidentified
GGVCPKILQRCRRDSDCPGACICQGNGYCCSGSD

SEQ ID NO: 320 - protein, 34 aa, unidentified
GGVCPRILKKCRRDSDCPGACICRGNGYCGSGSD

SEQ ID NO: 321 - protein, 30 aa, Momordica cochinchinensis
ERACPRILKKCRRDSDCPGECICKENGYCG

SEQ ID NO: 322 - protein, 30 aa, unidentified
QRACPRILKKCRRDSDCPGECICKGNGYCG

SEQ ID NO: 323 - protein, 30 aa, unidentified
QRACPRILKKCRRDSDCPGECICQGNGYCG

SEQ ID NO: 324 - protein, 31 aa, unidentified
QQRACPRILKKCRRDSDCPGECICKGNGYCG

SEQ ID NO: 325 - protein, 34 aa, unidentified
QRACPKILKRCRRDSDCPGACVCQDNGYCGSGGD

SEQ ID NO: 326 - protein, 34 aa, unidentified
QRACPRILKRCSRDSDCPGACVCQDNGYCGSRGD

SEQ ID NO: 327 - protein, 34 aa, unidentified
QRACPRILKRCRRDSDCPGACVCQGNGYCGSRGD

SEQ ID NO: 328 - protein, 34 aa, unidentified
QRACPRILKRCRRDSDCPGACVCQDNGYCGSGGD

SEQ ID NO: 329 - protein, 29 aa, unidentified
GICPRILMECKRDSDCLAQCVCKRQGYCG

SEQ ID NO: 330 - protein, 29 aa, unidentified
AICPRILVECKRDSDCPAQCICKRQGYCG

SEQ ID NO: 331 - protein, 28 aa, Momordica charantia
RSCPRIWMECTRDSDCMAKCICVAGHCG

SEQ ID NO: 332 - protein, 30 aa, Momordica charantia
ERRCPRILKQCKRDSDCPGECICMAHGFCG

SEQ ID NO: 333 - protein, 30 aa, Momordica charantia
ERGCPRILKQCKQDSDCPGECICMAHGFCG

SEQ ID NO: 334 - protein, 28 aa, Momordica charantia
RICPRIWMECKRDSDCMAQCICVDGHCG

SEQ ID NO: 335 - protein, 31 aa, Momordica charantia
EEERICPRIWMECKRDSDCMAQCICVDGHCG

SEQ ID NO: 336 - protein, 28 aa, Momordica charantia
RICPLIWMECKRDSDCLAQCICVDGHCG

SEQ ID NO: 337 - protein, 29 aa, Momordica charantia
ERICPLIWMECKRDSDCLAQCICVDGHCG

SEQ ID NO: 338 - protein, 30 aa, Momordica charantia
EERICPLIWMECKRDSDCLAQCICVDGHCG

SEQ ID NO: 339 - protein, 31 aa, Momordica charantia
EEERICPLIWMECKRDSDCLAQCICVDGHCG

SEQ ID NO: 340 - protein, 31 aa, Momordica charantia
EDERICPLIWMECKRDSDCLAQCICVDGHCG

(19) United States
(12) Patent Application Publication
(10) Pub. No.: US 2024/0092846 A1
(43) Pub. Date: Mar. 21, 2024

(54) ENGINEERED CYCLOTIDES WITH POTENT BROAD ANTIMICROBIAL ACTIVITY

(71) Applicant: University of Southern California, Los Angeles, CA (US)

(72) Inventors: Julio Camarero Palao, Los Angeles, CA (US); Paul Beringer, Los Angeles, CA (US); Mansour Dughbaj, Los Angeles, CA (US); Rajasekaran Ganesan, Los Angeles, CA (US)

(21) Appl. No.: 18/212,068

(22) Filed: Jun. 20, 2023

Related U.S. Application Data
(60) Provisional application No. 63/353,976, filed on Jun. 21, 2022.

(51) Int. Cl. C07K 14/47; A61P 31/04

(52) U.S. Cl. CPC C07K 14/4703; A61P 31/04; A61K 38/00; C07K 2317/73

(57) ABSTRACT

Disclosed herein are novel engineered cyclotides with effective broad-spectrum antibacterial activity against several ESKAPE bacterial strains and clinical isolates.

### CROSS-REFERENCE TO RELATED PATENT APPLICATIONS

This application claims priority under 35 U.S.C. § 119(e) of U.S. provisional application U.S. Ser. No. 63/353,976, filed Jun. 21, 2022, the contents of which are incorporated herein by reference.

### STATEMENT OF GOVERNMENT SUPPORT

This invention was made with government support under Grant Nos. R01GM113636 and R35GM132072 awarded by the National Institutes of Health (NIH) and National Institute of General Medical Sciences (NIGMS). The government has certain rights in the invention.

### SEQUENCE LISTING

The instant application contains a Sequence Listing which has been submitted electronically in XML format and is hereby incorporated by reference in its entirety. Said XML copy, created on Nov. 16, 2023, is named 064189-9502_SL.xml and is 314,420 bytes in size.

### BACKGROUND

The search for novel antimicrobial agents is intensifying, in response to the threat of microbial pathogens and the increasing development of drug resistance to current antibiotic therapeutics. According to the Centers for Disease Control and Prevention, the six ESKAPE (Enterococcus faecium, Staphylococcus aureus, Klebsiella pneumoniae, Acinetobacter baumanii, Pseudomonas aeruginosa, and Enterobacter species) bacterial species cause two-thirds of health care-associated infections (e.g. pneumonia, septicemia), leading to 99,000 deaths annually in the United States [1] A hallmark of these emerging difficult-to-treat clinical superbugs is their ability to “escape” the action of multiple traditional antibiotics, in part due to biofilm formation and mechanisms of drug resistance.[2] Antimicrobial peptides are essential host defense molecules found in a wide variety of species and are promising antibacterial therapeutic candidates.[3] Several hundreds of antimicrobial peptides have been identified in a variety of life forms ranging from bacteria, fungi, plants, amphibians, to mammals, including humans.[4] In mammals, cathelicidins, protegrins and defensins are the three of major types of host defense peptides.[5]

### SUMMARY OF THE DISCLOSURE

Preliminary studies have shown that 0-hairpin-containing antimicrobial peptides have potent antimicrobial activity and cell selectivity.[6] For example, the two-b-strand protegrin 1 (PG-1) (FIG. 1), an 18-amino-acid long peptide, is a prototypic antimicrobial cationic peptide of the protegrin family isolated from porcine leukocytes.[7] Protegrin PG-1 is smaller in size than a- and b-defensins but shows significant size and structural similarities with another family of antimicrobial peptides, the tachyplesins,[8] showing also sequence homology with the N-terminal region of a-defensins.[9] In solution, PG-1 forms a well-ordered antiparallel b-sheet structure stabilized by the presence of two disulfide bonds with disordered N- and C-termini.[10] The presence of the disulfide bonds is required to maintain potent antimicrobial activity.[11] PG-1 has been shown to disrupt anionic bacterial membranes and biofilms, showing also a wide range of in vivo immunomodulatory properties like inhibition of LPS and increasing neutrophil clearance.[6b, 6c] This distinct antimicrobial mechanism of action of PG-1 limits potential cross-resistance while providing synergy in combination with other locally produced host defense peptides and/or conventional antibiotics.[6d] The effectiveness of PG-1 in several different animal infection and inflammation models suggests that this type of peptide may represent a new class of antibiotic and immunomodulatory reagents.[12] However, their therapeutic use is currently limited by their high cytotoxicity, hemolytic activity and suboptimal biological stability.[13]

Applicant discloses an antimicrobial comprising a cyclotide backbone and a protegrin PG-1 polypeptide (PG-1). In one aspect, the PG-1 comprises, or consists essentially of, or yet further consists of the polypeptide N-X1GRLCYCRRRFCVCVGRX2-C(SEQ ID NO: 291), wherein “N” indicates the amino terminus and “C” indicates the carboxy terminus. In one aspect, X₁ and X₂ of PG-1 are the same or different and comprise 0 to 5 amino acids selected from G, R and L. In a further aspect, X₁ and X₂ are the same and are G or R. In another aspect, they are different and are G or R. In one aspect they are the same and are R or G.

In a further aspect, PG-1 comprises, or consists of, or consists essentially of the polypeptide of the group of RGGRLCYCRRRFCVCVGR (SEQ ID NO: 5); GGRLCYCRRRFCVCVGRR (SEQ ID NO: 6); GGCLCYCRRRFCVCVCRR (SEQ ID NO: 7); GGGRLCYCRRRFCVCVGRRG (SEQ ID NO: 8); or GRLCYCRRRFCVCVGR (SEQ ID NO: 9), or an equivalent of each thereof. In another aspect, PG-1 comprises, or consists of, or consists essentially of: the polypeptide of the group of RGGRLCYCRRRFCVCVGR (SEQ ID NO: 5); GGRLCYCRRRFCVCVGRR (SEQ ID NO: 6); GGCLCYCRRRFCVCVCRR (SEQ ID NO: 7); GGGRLCYCRRRFCVCVGRRG (SEQ ID NO: 8); or GRLCYCRRRFCVCVGR (SEQ ID NO: 9). In a further aspect, the PG-1 comprises or consists essentially of the polypeptide: GGRLCYCRRRFVCVGRR (SEQ ID NO: 292).

Also provided herein is an antimicrobial as described above, wherein the cyclotide backbone is selected from the group of SEQ ID NOs: 1 to 4 or 10 to 290 or a cyclotide from the Momordica spp plant, or an equivalent of each thereof, wherein the equivalent comprises a polypeptide that maintains a cystine-knot scaffold and head-to-tail cyclization but in which hypermutation of essentially all residues is permitted with the exception of the strictly conserved cysteine that comprise the knot. In another aspect, the cyclotide backbone is a selected from the group of SEQ ID NOs: 1 to 4, or an equivalent of each thereof, wherein the equivalent comprises a polypeptide that maintains a cysteine-knot scaffold and head-to-tail cyclization but in which hypermutation of essentially all residues is permitted with the exception of the strictly conserved cysteines that comprise the knot.

In one embodiment, the PG-1 polypeptide is grafted into any one of loops 1 to 6 of the cyclotide backbone. In another embodiment, the PG-1 polypeptide is grafted into loop 6 of the cyclotide backbone. The cyclotide comprises a molecular framework comprising a sequence of amino acids forming a cyclic backbone wherein the cyclic backbone comprises sufficient disulfide bonds to confer knotted topology on the molecular framework or part thereof. Examples of cyclic backbone polypeptides are now in the art and described herein.

The antimicrobials as described herein can further comprising a label or purification marker and/or a carrier, such as a pharmaceutically acceptable carrier.

Also provided is a plurality of antimicrobials as described herein that may be the same or different from each other. These can further comprise, consist essentially of, or consist of, a carrier such as a pharmaceutically acceptable carrier.

In another aspect, the carrier further comprises, or consists essentially of, or yet further consists of, an additional antibiotic or antimicrobial.

Yet further provided are isolated polynucleotides encoding the antimicrobials as described herein as well as a complement of each. In one aspect, the isolated polynucleotide or complement further comprises a label or a purification marker and can be combined with a carrier, such as a pharmaceutically acceptable carrier.

The antimicrobials of this disclosure are useful in a variety of in vitro and in vivo methods. In one aspect, the antimicrobials are administered to a subject in need thereof to inhibit the growth of a microorganism or treat an infection by the microorganism in a subject in need thereof. In another aspect, the antimicrobial is provided to inhibit the growth of a microorganism or a cell containing same by contacting the cell or organism with an effective amount of the antimicrobial, the plurality, the composition, polynucleotide, or cell as described herein. Contacting can be in vitro or in vivo.

The compositions can be administered to an animal or mammal by a treating veterinarian or to a human patient by a treating physician.

In one aspect, provided is a method for one or more of the following: inhibiting, preventing or treating a microbial infection that produces a biofilm in a subject, treating a condition characterized by the formation of a biofilm in a subject. The method comprises, or consists essentially of, or yet further consists of administering to the subject one or more of: a composition as disclosed herein, an antimicrobial as disclosed herein, a polynucleotide as disclosed herein, a vector as disclosed herein, or a host cell as disclosed herein. In some aspects relating to any method(s) as disclosed herein, optionally comprising an administration step an additional antimicrobial or biofilm disrupting agent. In some aspects, the condition characterized by the formation of a biofilm is selected from the group consisting of: chronic non-healing wounds, Burkholderia, venous ulcers, diabetic foot ulcers, ear infections, sinus infections, urinary tract infections, gastrointestinal tract ailments, pulmonary infections, respiratory tract infections, cystic fibrosis, chronic obstructive pulmonary disease, catheter-associated infections, indwelling devices associated infections, infections associated with implanted prostheses, osteomyelitis, cellulitis, abscesses, and periodontal disease.

When practiced in vivo in non-human animal such as a chinchilla, the method provides a pre-clinical screen to identify agents that can be used alone or in combination with other agents to disrupt biofilms.

Kits to prepare and use the antimicrobials and further provided herein optionally comprising instructions for use.

### BRIEF DESCRIPTION OF THE DRAWINGS

FIG. 1. Left and Right Panels: Scheme depicting the approach used to design exemplary MCo-PG antimicrobial cyclotides. Left Panel: A circular permuted version of porcine protegrin PG-1, where the original Arg[1] residue in PG-1 was moved to its C-terminus, was grafted into onto of cyclotide loop 6 of cyclotide MCoTI-I between Gly[1] and Ser[33] residues. Right Panel: The backbone cyclized structure of the cyclotide is shown a connecting bond in gray. Cys residues are shown in color and disulfide bonds are indicated in connecting lines below the peptides. The ribbon structures of cyclotide MCoTI-II (PDB: 1IB9)[33] and porcine protegrin PG-1 (PDB: 1PG1)[7b] are shown for reference (bottom figure of Left Panel). Figure discloses SEQ ID NOS 297-302, respectively, in order of appearance.

FIGS. 2A-2C: Chemical synthesis and characterization of exemplary cyclotide MCo-PG2. (FIG. 2A, 3 panels) Analytical HPLC traces of for the linear thioester precursor, GSH-induced cyclization/folding crude after 72 h and purified cyclotide. An asterisk indicates the peak of the desired peptide. (FIG. 2B) ES-MS characterization of pure MCo-PG2. The expected average molecular weight is shown in parenthesis. (FIG. 2C) Chemical shifts differences of the backbone, H′ and H^{α} protons between the common sequence (residues 1 through 34) of MCoTI-I [24] and MCo-PG2 (Table 4).

FIGS. 3A-3C: Cytotoxic activities of cyclotide MCo-PG2. (FIG. 3A) Bactericidal activity of PG-1 against log-phase P. aeruginosa PAO1. P. aeruginosa was grown to log phase, and aliquots were treated with compounds at incremental concentrations relative to MICs, from to 0.25×MIC to 16×MIC. (FIG. 3B) Hemolytic activity of protegrin PG-1 and cyclotide MCo-PG2. Hemolytic activity was determined using human erythrocytes in PBS. Peptide concentrations causing 50% hemolysis (HC₅₀) were derived from the dose-response curves. (FIG. 3C) Cytotoxic profile of protegrin PG-1 and cyclotide MCo-PG2 to various mammalian cells (A549 and HEK293T). Cells were treated with increasing concentrations of the corresponding peptides. Cell viability was assessed by using the MTT assay. Cyclotide MCoTI-I was used as control. Data are mean±SEM for experiments performed in triplicate.

FIG. 4: Evaluation of exemplary cyclotide MCo-PG2 against P. aeruginosa (Schroeter) Migula (ATCC 27853) in a P. aeruginosa-induced bacterial peritonitis model.[29] P. aeruginosa was administered to mice by intraperitoneal injection 1.5×10⁷ colony forming units (CFU) per mouse. The animals were then immediately treated by intraperitoneal injection with PG-1 (5 mg/kg) and MCo-PG2 (10 or 25 mg/kg). Colistin (15 mg/kg) and PBS were used as positive and negative controls. The numbers of surviving mice were determined daily for 7 days. Single-dose administrations of MCo-PG2 (10 mg/kg, 1.8 μmol/kg; 25 mg/kg, 4.5 μmol/kg) were associated with a high survival rate of septic mice (Hazard ratio (HR): 11.4 and 20.8 respectively, p<0.001) comparable to treatments with PG-1 (5 mg/kg, 2.3 μmol/kg) and 15 mg/kg colistin (15 mg/kg, 12.3 μmol/kg) (HR: 24.8, p<0.001).

FIG. 5: Analytical reverse-phase C18-HPLC traces and ES-MS spectra of MCo-PG linear precursor thioesters, cyclization/folding crudes and purified folded cyclotides. HPLC analysis was performed using a linear gradient of 0-70% solvent B over 30 minutes. The asterisk denotes the peak of the corresponding product.

FIG. 6: Overlaid 2D ¹H-¹H TOCSY spectra of MCoTI-I (red) and MCo-PG2 (blue) in 20% (v/v) d4-MeOD and 80% (v/v) 5 mM potassium phosphate buffer, pH 6.0.

FIG. 7: Amide-amide region of the 2D ¹ H-¹H NOESY (150 ms mixing time) spectrum for MCo-PG2 in 20% (v/v) d4-MeOD, 80% (v/v) 5 mM potassium phosphate buffer at pH 6.0. Long range nuclear Overhauser effect, NOE, cross peaks are indicated for amide protons of Y39/V46 and L37/V48. These H′-H′ connectivities were also observed in PG-1 (11). Other long-range NOEs detected in PG-1 that were not observed in MCo-PG2 include: H′R41/H′F44 (R41 signal broadened beyond detection), H′R41/H′R43 (R41 and R43 signals broadened beyond detection), H^{α} R36/H^{α} G49 (NOEs may be missing due to water suppression) H^{α} C40/H^{α} C45 (NOEs may be missing due to water suppression).

FIG. 8: Stability of cyclotides MCo-PG2, MCoTI-I, and protegrin PG-1 to human serum at 37° C. Linearized reduced cyclotide was used as control for serum activity. Undigested peptide was quantified by HPLC-MS/MS.

FIG. 9: Toxicological data for antimicrobial cyclotide MCo-PG2 and PG-1. The MTD was determined using two different endpoints: weight loss and clinical scoring. Clinical scores were evaluated through activity, appearance and body condition, similar to previous published literature (42).

FIG. 10: Analytical reverse-phase C18-HPLC traces and ES-MS spectra for reduced linear PG-1, cyclization/folding crude and purified PG-1. HPLC analysis was performed using a linear gradient of 0-70% solvent B over 30 minutes. The peaks marked with “*” denotes the expected product. The peak marked with “#” corresponds a non-peptide impurity from the TFA acidolytic cocktail. Expected molecular weights are shown in parenthesis.

FIGS. 11A-11B show exemplary cyclotides from the Momordica spp plants. (Reproduced from Mahatmanto et al. (2014) Mol. Bio. And Evol. 32(2):392-405). Figure discloses SEQ ID NOS 303-340, respectively, in order of columns.

FIG. 12 depicts an inoculation scheme for use of the cyclotides of this disclosure.

### DETAILED DESCRIPTION

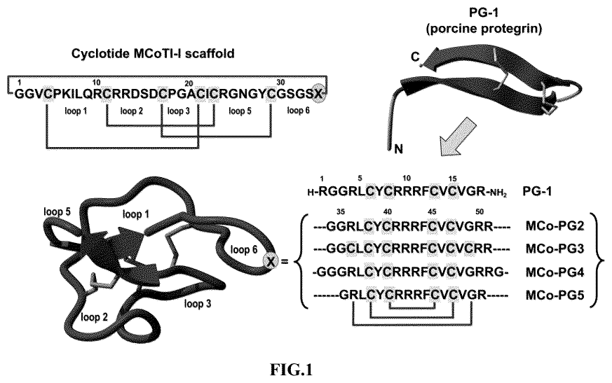

Cyclotides are fascinating micro-proteins (≈30 residues long) present in plants from different families including Violaceae, Rubiaceae, Cucurbitaceae, and Fabaceae families, among others.[14] They have shown a broad array of biological activities such as protease inhibitory, anti-microbial, insecticidal, cytotoxic, anti-HIV, and hormone-like activities.[15] They share a unique head-to-tail circular knotted topology of three disulfide bridges, with one disulfide penetrating through a macrocycle formed by the two other disulfides and inter-connecting peptide backbones, forming what is called a cystine knot topology[15a] (FIG. 1). Cyclotides can be considered as natural combinatorial peptide framework structurally constrained by the cystine-knot scaffold and head-to-tail cyclization but in which hypermutation of essentially all residues is permitted with the exception of the strictly conserved cysteines that comprise the cystine knot.[15a, 15b] Cyclotides are characterized by possessing a remarkable stability due to the presence of a backbone cyclized cystine knot topology, a small size making them readily accessible to chemical synthesis [16] and heterologous expression,[17] and exceedingly tolerant to sequence variations and molecular grafting.[15b] In addition cyclotides have shown to be orally active,[18] and capable of crossing cell membranes[19] to efficiently target intracellular targets in vivo.[20] Altogether, these features make the cyclotide scaffold an excellent molecular framework for the design of novel peptide-based therapeutics,[15b, 21] making them ideal substrates for molecular grafting of biological peptide epitopes.[15a]

By using a topologically modified sequence of PG-1, Applicant provides herein for the first time a novel engineered cyclotide with effective broad-spectrum antibacterial activity against several ESKAPE bacterial strains and clinical isolates. One exemplary antibacterial cyclotide showed little hemolytic activity and was extremely stable in serum. In addition, this cyclotide was able to provide in vivo protection in a murine model of P. aeruginosa peritonitis. These results highlight for the first time the potential of the cyclotide scaffold for the development of novel therapeutic leads for the treatment of bacteremia.

### Definitions

This disclosure references various publications, patents and published patent specifications by an identifying citation or an Arabic number. The full citations for the disclosures referenced by an Arabic number are found immediately preceding the claims. The disclosures of these publications, patents and published patent specifications are hereby incorporated by reference into the present disclosure in their entirety to more fully describe the state of the art to which this invention pertains.

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; Animal Cell Culture (R. I. Freshney, ed. (1987)); Zigova, Sanberg and Sanchez-Ramos, eds. (2002) Neural Stem Cells.

All numerical designations, e.g., pH, temperature, time, concentration, and molecular weight, including ranges, are approximations which are varied (+) or (−) by increments of 0.1 or 1 where appropriate. It is to be understood, although not always explicitly stated that all numerical designations are preceded by the term “about”. The term “about” also includes the exact value “X” in addition to minor increments of “X” such as “X+0.1 or 1” or “X−0.1 or 1,” where appropriate. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to proteins, polypeptides, cells, nucleic acids, such as DNA or RNA, refers to molecules separated from other proteins, polypeptides, cells, nucleic acids, such as DNA or RNA, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together. A recombinant polynucleotide is a polynucleotide created or replicated using techniques (chemical or using host cells) other than by a cell in its native environment.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

Cells,” “host cells” or “recombinant host cells” are terms used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

“Amplify” “amplifying” or “amplification” of a polynucleotide sequence includes methods such as traditional cloning methodologies, PCR, ligation amplification (or ligase chain reaction, LCR) or other amplification methods. These methods are known and practiced in the art. See, e.g., U.S. Pat. Nos. 4,683,195 and 4,683,202 and Innis et al. (1990) Mol. Cell Biol. 10(11):5977-5982 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “genotype” refers to the specific allelic composition of an entire cell, a certain gene or a specific polynucleotide region of a genome, whereas the term “phenotype’ refers to the detectable outward manifestations of a specific genotype.

As used herein, the term “gene” or “recombinant gene” refers to a nucleic acid molecule comprising an open reading frame and including at least one exon and (optionally) an intron sequence. A gene may also refer to a polymorphic or a mutant form or allele of a gene.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on May 21, 2008. Biologically equivalent polynucleotides are those having the above-noted specified percent homology and encoding a polypeptide having the same or similar biological activity.

In one aspect, the term “equivalent” as it refers to polypeptides, proteins, or polynucleotides refers to polypeptides, oligopeptides, proteins, or polynucleotides, respectively having a sequence having a certain degree of homology or identity with the reference sequence of the polypeptides, proteins, or polynucleotides (or complement thereof when referring to polynucleotides). A homolog of a double stranded nucleic acid is intended to include nucleic acids having a nucleotide sequence that has a certain degree of homology with or with the complement thereof. In one aspect, homologs of nucleic acids are capable of hybridizing to the nucleic acid or complement thereof. In one aspect, an equivalent has at least 70%, or at least 75% or at least 80%, or at least 85%, or at least 90%, or at least 95%, or at least 97%, or at least 98%, sequence identity to the reference polynucleotide or polypeptide. The term “equivalent” may also refer to a cyclotide equivalent that comprises a polypeptide that maintains a cysteine-knot scaffold and head-to-tail cyclization but in which hypermutation of essentially all residues is permitted with the exception of the strictly conserved cysteines that comprise the knot.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

As used herein, the term “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “polynucleotide” and “oligonucleotide” are used interchangeably and refer to a polymeric form of nucleotides of any length, either deoxyribonucleotides or ribonucleotides or analogs thereof. Polynucleotides can have any three dimensional structure and may perform any function, known or unknown. The following are non limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non nucleotide components. A polynucleotide can be further modified after polymerization, such as by conjugation with a labeling component. The term also refers to both double and single stranded molecules. Unless otherwise specified or required, any embodiment of this invention that is a polynucleotide encompasses both the double stranded form and each of two complementary single stranded forms known or predicted to make up the double stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide,” “oligopeptide,” “protein,” and “peptide” are used interchangeably and refer to a polymer of amino acids of any length, held together by amide bonds. Polypeptides can have any primary, secondary, tertiary, or quaternary structure and may perform any function, known or unknown. A polypeptide can comprise standard amino acids, modified amino acids, unnatural amino acids, enantiomers, and analogs thereof. If present, modifications to the amino acids can be imparted before or after assembly, synthesis, or translation of the polypeptide. A polypeptide can be further modified by conjugation with a labeling component.

As used herein, the term “carrier” encompasses any of the standard carriers, such as a phosphate buffered saline solution, buffers, water, and emulsions, such as an oil/water or water/oil emulsion, and various types of wetting agents. The compositions also can include stabilizers and preservatives. For examples of carriers, stabilizers and adjuvants, see Sambrook and Russell (2001), supra. Those skilled in the art will know many other suitable carriers for binding polynucleotides, or will be able to ascertain the same by use of routine experimentation. In one aspect of the invention, the carrier is a buffered solution such as, but not limited to, a PCR buffer solution.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

The term “express” refers to the production of a gene product. When used in reference to a cancer cell or a tumor cell, “express” may also refer to an increased or abnormal level of production of a gene product by the cancer or tumor cell relative to normal cells.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

A “gene product” or alternatively a “gene expression product” refers to the RNA generated when a gene is transcribed or the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, a “vector” is a vehicle for transferring genetic material into a cell. Examples of such include, but are not limited to plasmids and viral vectors. A viral vector is a virus that has been modified to transduct genetic material into a cell. A plasmid vector is made by splicing a DNA construct into a plasmid. As is apparent to those of skill in the art, the appropriate regulatory elements are included in the vectors to guide replication and/or expression of the genetic material in the selected host cell.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral Vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, CA) and Promega Biotech (Madison, WI). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmic vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacteria produces proteins to confer its antibiotic resistance, it can also be induced to produce large amounts of proteins from the inserted gene. This is a cheap and easy way of mass-producing a gene or the protein it then codes for.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, yeast, higher plant, insect and mammalian cells. Non-limiting examples include simian, bovine, ovine, porcine, murine, rats, canine, equine, feline, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to prokaryotic Cyanobacteria, Bacillus bacteria, E. coli bacterium, and Salmonella bacterium.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′ OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optional contain detectable labels and are exemplified and described herein.

As used herein, the term “detectable label” intends a directly or indirectly detectable compound or composition (other than a naturally occurring polynucleotide in its natural environment) that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are known in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarray system that can dispense from 12 to 64 spots onto multiple glass slides (Intelligent Bio-Instruments); Molecular Biology Workstation and NanoChip (Nanogen, Inc.); a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or “microarrays” are also described in U.S. Patent Publication Nos.: 2007/0111322; 2007/0099198; 2007/0084997; 2007/0059769 and 2007/0059765 and U.S. Pat. Nos. 7,138,506; 7,070,740 and 6,989,267.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. In general, one will desire to administer an amount of the compound that is effective to achieve a serum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatment can include prophylaxis or in one aspect, can exclude prophylaxis.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets. The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

“Administration” can be provided in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

As used herein, a biological sample, or a sample, can be obtained from a subject, cell line or cultured cell or tissue. Exemplary samples include, but are not limited to, cell sample, tissue sample, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In one embodiment, the biological sample is suspect of having a biofilm. In another embodiment, the biological sample comprise a biofilm.

A “biofilm” intends an organized community of microorganisms that at times adhere to the surface of a structure, that may be organic or inorganic, together with the polymers such as DNA that they secrete, release and/or become available in the extracellular milieu due to bacterial lysis. The biofilms are very resistant to microbiotics and antimicrobial agents. They live on gingival tissues, teeth and restorations, causing caries and periodontal disease, also known as periodontal plaque disease. They also cause chronic middle ear infections. Biofilms can also form on the surface of dental implants, stents, catheter lines and contact lenses. They grow on pacemakers, heart valve replacements, artificial joints and other surgical implants. The Centers for Disease Control) estimate that over 65% of nosocomial (hospital-acquired) infections are caused by biofilms. They cause chronic vaginal infections and lead to life-threatening systemic infections in people with hobbled immune systems. Biofilms also are involved in numerous diseases. For instance, cystic fibrosis patients have Pseudomonas infections that often result in antibiotic resistant biofilms.

A “biofilm associated disease” intends a disease or condition in which a biofilm is present at some point in the disease state. Non-limiting examples include: chronic non-healing wounds, Burkholderia, venous ulcers, diabetic foot ulcers, ear infections, sinus infections, urinary tract infections, cardiac disease, gastrointestinal tract ailments, pulmonary infections, respiratory tract infections, cystic fibrosis, chronic obstructive pulmonary disease, catheter-associated infections, indwelling devices associated infections, infections associated with implanted prostheses, osteomyelitis, cellulitis, abscesses, and periodontal disease. In one aspect it is cystic fibrosis. In another aspect it is inner ear infections.

As used herein, the ESKAPE pathogens include Enterococcus faecium, Staphylococcus aureus, Klebsiella pneumoniae, Acinetobacter baumannii, Pseudomonas aeruginosa, and Enterobacter species. These pathogens are the leading cause of nosocomial infections throughout the world.

A “control” is an alternative subject or sample used in an experiment for comparison purpose. A control can be “positive” or “negative.”

### Antimicrobial Cyclotides

Cyclotides are small globular microproteins (ranging from 28 to 37 amino acids) with a unique head-to-tail cyclized backbone, which is stabilized by disulfide bonds forming a cystine-knot motif. This cyclic cystine-knot (CCK) framework provides a rigid molecular platform with exceptional stability towards physical, chemical and biological degradation. These micro-proteins can be considered natural combinatorial peptide libraries structurally constrained by the cystine-knot scaffold and head-to-tail cyclization, but in which hypermutation of essentially all residues is permitted with the exception of the strictly conserved cysteines that comprise the knot. Furthermore, naturally occurring cyclotides have shown to possess various pharmacologically relevant activities, and have been reported to cross cell membranes. Altogether, these features make the cyclotide scaffold an excellent molecular framework for the design of novel peptide-based therapeutics, making them ideal substrates for molecular grafting of biological peptide epitopes.

The construction of a modified cyclotide is known in the art and has been described previously (see WO 2011/005598 and U.S. Pat. No. 10,988,522, which are incorporated herein for all purposes). Synthesis of peptides useful in the methods and compositions of the disclosure are also described herein and known in the art. Exemplary cyclotides are provided herein.

The preparation of a cyclotide may also entail the generation of a linear peptide that contains the desired cyclotide in a linear form, flanked by two peptide fragments that have affinity to each other so as to be capable of bringing two ends of the linear cyclotide together, facilitating cyclization. Accordingly, the present disclosure provides a polypeptide precursor for generating a cyclotide.

In one aspect, provided herein is an antimicrobial comprising a cyclotide backbone and an protegrin PG-1 polypeptide (PG-1). In one aspect, the PG-1 comprises, or consists essentially of, or yet further consists of the polypeptide N-X1GRLCYCRRRFCVCVGRX2-C(SEQ ID NO: 291). In one aspect, X₁ and X₂ of PG-1 are the same or different an comprise 0 to 5 amino acids selected from G, R and L. In a further aspect, X₁ and X₂ are the same and are G or R. In another aspect, they are different and are G or R. In one aspect they are the same and are R or G. In a further aspect, PG-1 comprises, or consists of, or consists essentially of: the polypeptide of the group of RGGRLCYCRRRFCVCVGR (SEQ ID NO: 5); GGRLCYCRRRFCVCVGRR (SEQ ID NO: 6); GGCLCYCRRRFCVCVCRR (SEQ ID NO: 7); GGGRLCYCRRRFCVCVGRRG (SEQ ID NO: 8); or GRLCYCRRRFCVCVGR (SEQ ID NO: 9), or an equivalent of each thereof. In another aspect, PG-1 comprises, or consists of, or consists essentially of: the polypeptide of the group of RGGRLCYCRRRFCVCVGR (SEQ ID NO: 5); GGRLCYCRRRFCVCVGRR (SEQ ID NO: 6); GGCLCYCRRRFCVCVCRR (SEQ ID NO: 7); GGGRLCYCRRRFCVCVGRRG (SEQ ID NO: 8); or GRLCYCRRRFCVCVGR (SEQ ID NO: 9). In a further aspect, the PG-1 comprises or consists essentially of the polypeptide: GGRLCYCRRRFVCVGRR (SEQ ID NO: 292).

Also provided herein is an antimicrobial as described above, wherein the cyclotide backbone is selected from the group of SEQ ID NOs: 1 to 4 or 10 to 290 or a cyclotide from the Momordica spp plant, or an equivalent of each thereof, wherein the equivalent comprises a polypeptide that maintains a cystine-knot scaffold and head-to-tail cyclization but in which hypermutation of essentially all residues is permitted with the exception of the strictly conserved cysteine that comprise the knot. In another aspect, the cyclotide backbone is a selected from the group of SEQ ID NOs: 1 to 4, or an equivalent of each thereof, wherein the equivalent comprises a polypeptide that maintains a cystine-knot scaffold and head-to-tail cyclization but in which hypermutation of essentially all residues is permitted with the exception of the strictly conserved cysteines that comprise the knot.

Reference herein to a “cyclotide backbone” includes a molecule comprising a sequence of amino acid residues or analogues thereof without free amino and carboxy termini. The cyclic backbone of the disclosure comprises sufficient disulfide bonds, or chemical equivalents thereof, to confer a knotted topology on the three-dimensional structure of the cyclic backbone. The term “cyclotide” as used herein refers to a peptide comprising a cyclic cystine knot motif defined by a cyclic backbone, at least two but preferably at least three disulfide bonds and associated beta strands in a particular knotted topology. The knotted topology involves an embedded ring formed by at least two backbone disulfide bonds and their connecting backbone segments being threaded by a third disulfide bond. However, a disulfide bond may be replaced or substituted by a mimic of a disulfide bond such as 1,4-disubstituted 1,2,3-triazoles introduced through copper (I)-catalyzed azide-alkyne cycloaddition (CuAAC) or 1,5-disubstituted 1,2,3-triazoles introduced through a ruthenium (II)-catalyzed method (RuAAC), or another form of bonding such as an amide bond, thioethers, diselenide bond, triazoles, hydrocarbon-based bridges, ionic bonds, hydrogen bonds, or hydrophobic bonds. In some embodiments, a cyclotide backbone comprises between about 20 and about 100, between about 25 and about 50, between about 27 and about 42, between about 30 and about 40, between about 32 and about 38, or between about 28 and 37 amino acid residues.

In some embodiments, the cyclotide backbone is comprised of, or alternatively consists essentially of MCoTI-I. The sequence of MCoTI-I is described FIG. 1. In one aspect, MCoTI-I comprises the sequence GGVCPKILQRCRRDSDCPGACICRGNGYCGSGSD (SEQ ID NO: 1), or an equivalent thereof. In another aspect, the sequence comprises GGBCPKILQRCRRDSDCPGACICRGAGYCGSGSD (SEQ ID NO: 2), or an equivalent thereof. In some aspects, two residues are removed from the carboxy terminal end so that the sequence of MCoTI-I comprises or consists essentially of GGBCPKILQRCRRDSDCPGACICRGAGYCGSG (SEQ ID NO: 3) or GGVCPKILQRCRRDSDCPGACICRGNGYCGSG (SEQ ID NO: 4). In further aspect, residue B in the above sequences represents asparagine or aspartic acid. In another aspect, the cyclotide backbone comprises a polypeptide or an equivalent thereof comprising a SEQ ID of any one of 10 to 290 or those shown in Table 6 below or in FIG. 11A or 11B.

Additional cyclotides useful in the peptides, methods, and compositions described herein are known in the art and non-limiting examples include, the cyclotides listed in Table 1 below. In some embodiments, the cyclotide backbone is derived from, comprises, or alternatively consists essentially of one or more of the sequences listed in Table 6 (SEQ ID NOS: 10 to 290). In some aspects, residue X in the amino acid sequences of Table 6 represents one or more unnatural amino acids.

TABLE 6

| | SEQ | |
|---|---|---|
| | ID | Cyclotide Backbone |
| Parental Cyclotide | NO: | Sequence |
| kalata_B1 | 10 | GLPVCGETCVGGTCN |
| | | TPGCTCSWPVCTRN |
| cycloviolacin_O1 | 11 | GIPCAESCVYIPCTV |
| | | TALLGCSCSNRVCYN |
| kalata_B2 | 12 | GLPVCGETCFGGTCN |
| | | TPGCSCTWPICTRD |
| Palicourein | 13 | GDPTFCGETCRVIPV |
| | | CTYSAALGCTCDDRS |
| | | DGLCKRN |
| vhr1 | 14 | GIPCAESCVWIPCTV |
| | | TALLGCSCSNKVCYN |
| tricyclon_A | 15 | GGTIFDCGESCFLGT |
| | | CYTKGCSCGEWKLCY |
| | | GTN |
| circulin_A | 16 | GIPCGESCVWIPCIS |
| | | AALGCSCKNKVCYRN |
| N-KB1-C | 17 | GLPVCGETCVGGTCN |
| | | TPGCTCSWPVCTRN |
| Ac-KB1-C | 18 | GLPVCGETCVGGTCN |
| | | TPGCTCSWPVCTRN |
| N-KB1-Am | 19 | GLPVCGETCVGGTCN |
| | | TPGCTCSWPVCTRN |
| Ac-KB1-Am | 20 | GLPVCGETCVGGTCN |
| | | TPGCTCSWPVCTRN |
| Ac-[desGly]-KB1-Am | 22 | LPVCGETCVGGTCNT |
| | | PGCTCSWPVCTRN |
| kalata_b1-1 | 23 | TCVGGTCNTPGCTCS |
| | | WPVCTRNLPVCG |
| kalata_b1-2 | 24 | GTCNTPGCTCSWPVC |
| | | TRNGLPVCGETCVG |
| kalata_b1-3 | 25 | GCTCSWPVCTRNGLP |
| | | VCGETCVGGTCN |
| kalata_b1-4 | 26 | CSWPVCTRNGLPVCG |
| | | ETCVGGTCNTPGC |
| kalata_b1-5 | 27 | VCTRNGLPVCGETCV |
| | | GGTCNTPGCTCS |
| kalata_b1-6a | 28 | VCGETCVGGTCNTPG |
| | | CTCSWPVCT |
| kalata_b1-6b | 29 | RNGLPVCGETCVGGT |
| | | CNTPGCTCSWPVCT |
| cycloviolacin_O2 | 30 | GIPCGESCVWIPCIS |
| | | SAIGCSCKSKVCYRN |
| des(24-28)kB1 | 31 | VCGETCVGGTCNTPG |
| | | CTCSWPVCT |
| [Ala1,15]kB1 | 32 | GLPVAGETCVGGTCN |
| | | TPGATCSWPVCTRN |
| kalata_B6 | 33 | GLPTCGETCFGGTCN |
| | | TPGCSCSSWPICTRN |
| kalata_B3 | 34 | GLPTCGETCFGGTCN |
| | | TPGCTCDPWPICTRD |
| kalata_B7 | 35 | GLPVCGETCTLGTCY |
| | | TQGCTCSWPICKRN |
| cycloviolacin_O8 | 36 | GTLPCGESCVWIPCI |
| | | SSVVGCSCKSKVCYK |
| | | N |
| cycloviolacin_O11 | 37 | GTLPCGESCVWIPCI |
| | | SAVVGCSCKSKVCYK |
| | | N |
| kalata_B4 | 38 | GLPVCGETCVGGTCN |
| | | TPGCTCSWPVCTRD |
| vodo_M | 39 | GAPICGESCFTGKCY |
| | | TVQCSCSWPVCTRN |
| cyclopsychotride_A | 40 | SIPCGESCVFIPCTV |
| | | TALLGCSCKSKVCYK |
| | | N |
| cycloviolacin_H1 | 41 | GIPCGESCVYIPCLT |
| | | SAIGCSCKSKVCYRN |
| cycloviolacin_O9 | 42 | GIPCGESCVWIPCLT |
| | | SAVGCSCKSKVCYRN |
| vico_A | 43 | GSIPCAESCVYIPCF |
| | | TGIAGCSCKNKVCYY |
| | | N |
| vitri_A | 44 | GIPCGESCVWIPCIT |
| | | SAIGCSCKSKVCYRN |
| kalata_S | 45 | GLPVCGETCVGGTCN |
| | | TPGCSCSWPVCTRN |
| cycloviolacin_O12 | 46 | GLPICGETCVGGTCN |
| | | TPGCSCSWPVCTRN |
| vodo_N | 47 | GLPVCGETCTLGKCY |
| | | TAGCSCSWPVCYRN |
| vico_B | 48 | GSIPCAESCVYIPCI |
| | | TGIAGCSCKNKVCYY |
| | | N |
| kalata_B1_Ila | 49 | GLPVCGETCVGGTCN |
| | | TPGCTCSWPVCTRN |
| Hypa_A | 50 | GIPCAESCVYIPCTI |
| | | TALLGCSCKNKVCYN |
| circulin_B | 51 | GVIPCGESCVFIPCI |
| | | STLLGCSCKNKVCYR |
| | | N |
| circulin_C | 52 | GIPCGESCVFIPCIT |
| | | SVAGCSCKSKVCYRN |
| circulin_D | 53 | KIPCGESCVWIPCVT |
| | | SIFNCKCENKVCYHD |
| circulin_E | 54 | KIPCGESCVWIPCLT |
| | | SVFNCKCENKVCYHD |
| circulin_F | 55 | AIPCGESCVWIPCIS |
| | | AAIGCSCKNKVCYR |
| cycloviolacin_O4 | 56 | GIPCGESCVWIPCIS |
| | | SAIGCSCKNKVCYRN |
| cycloviolacin_O3 | 57 | GIPCGESCVWIPCLT |
| | | SAIGCSCKSKVCYRN |
| cycloviolacin_O5 | 58 | GTPCGESCVWIPCIS |
| | | SAVGCSCKNKVCYKN |
| cycloviolacin_O6 | 59 | GTLPCGESCVWIPCI |
| | | SAAVGCSCKSKVCYK |
| | | N |
| cycloviolacin_O7 | 60 | SIPCGESCVWIPCTI |
| | | TALAGCKCKSKVCYN |
| cycloviolacin_O10 | 61 | GIPCGESCVYIPCLT |
| | | SAVGCSCKSKVCYRN |
| kalata_B5 | 62 | GTPCGESCVYIPCIS |
| | | GVIGCSCTDKVCYLN |
| varv_peptide_B | 63 | GLPVCGETCFGGTCN |
| | | TPGCSCDPWPMCSRN |
| varv_peptide_C | 64 | GVPICGETCVGGTCN |
| | | TPGCSCSWPVCTRN |
| varv_peptide_D | 65 | GLPICGETCVGGSCN |
| | | TPGCSCSWPVCTRN |
| varv_peptide_F | 66 | GVPICGETCTLGTCY |
| | | TAGCSCSWPVCTRN |
| varv_peptide_G | 67 | GVPVCGETCFGGTCN |
| | | TPGCSCDPWPVCSRN |
| varv_peptide_H | 68 | GLPVCGETCFGGTCN |
| | | TPGCSCETWPVCSRN |
| cycloviolin_A | 69 | GVIPCGESCVFIPCI |
| | | SAAIGCSCKNKVCYR |
| | | N |
| cycloviolin_B | 70 | GTACGESCYVLPCFT |
| | | VGCTCTSSQCFKN |
| cycloviolin_C | 71 | GIPCGESCVFIPCLT |
| | | TVAGCSCKNKVCYRN |
| cycloviolin_D | 72 | GFPCGESCVFIPCIS |
| | | AAIGCSCKNKVCYRN |
| violapeptide_1 | 73 | GLPVCGETCVGGTCN |
| | | TPGCSCSRPVCTXN |
| vh1-1 | 74 | SISCGESCAMISFCF |
| | | TEVIGCSCKNKVCYL |
| | | N |
| Vontr_Protein | 75 | ALETQKPNHLEEALV |
| | | AFAKKGNLGGLP |
| hcf-1 | 76 | GIPCGESCHYIPCVT |
| | | SAIGCSCRNRSCMRN |
| htf-1 | 77 | GIPCGDSCHYIPCVT |
| | | STIGCSCTNGSCMRN |
| Oantr_protein | 78 | GVKSSETTLMFLKEM |
| | | QLKLP |
| vh1-2 | 79 | GLPVCGETCFTGTCY |
| | | TNGCTCDPWPVCTRN |
| cycloviolacin_H3 | 80 | GLPVCGETCFGGTCN |
| | | TPGCICDPWPVCTRN |
| cycloviolacin_H2 | 81 | SAIACGESCVYIPCF |
| | | IPGCSCRNRVCYLN |
| Hyfl_A | 82 | SISCGESCVYIPCTV |
| | | TALVGCTCKDKVCYL |
| | | N |
| Hyfl_B | 83 | GSPIQCAETCFIGKC |
| | | YTEELGCTCTAFLCM |
| | | KN |
| Hyfl_C | 84 | GSPRQCAETCFIGKC |
| | | YTEELGCTCTAFLCM |
| | | KN |
| Hyfl_D | 85 | GSVPCGESCVYIPCF |
| | | TGIAGCSCKSKVCYY |
| | | N |
| Hyfl_E | 86 | GEIPCGESCVYLPCF |
| | | LPNCYCRNHVCYLN |
| Hyfl_F | 87 | SISCGETCTTFNCWI |
| | | PNCKCNHHDKVCYWN |
| Hyfl_G_(partial) | 88 | CAETCVVLPCFIVPG |
| | | CSCKSSVCYFN |
| Hyfl_H_(partial) | 89 | CAETCIYIPCFTEAV |
| | | GCKCKDKVCYKN |
| Hyfl_I | 90 | GIPCGESCVFIPCIS |
| | | GVIGCSCKSKVCYRN |
| Hyfl_J | 91 | GIACGESCAYFGCWI |
| | | PGCSCRNKVCYFN |
| Hyfl_K | 92 | GTPCGESCVYIPCFT |
| | | AVVGCTCKDKVCYLN |
| Hyfl_L | 93 | GTPCAESCVYLPCFT |
| | | GVIGCTCKDKVCYLN |
| Hyfl_M | 94 | GNIPCGESCIFFPCF |
| | | NPGCSCKDNLCYYN |
| Hyfl_N_(partial) | 95 | CGETCVILPCISAAL |
| | | GCSCKDTVCYKN |
| Hyfl_O_(partial) | 96 | CGETCVIFPCISAAF |
| | | GCSCKDTVCYKN |
| Hyfl_P | 97 | GSVPCGESCVWIPCI |
| | | SGIAGCSCKNKVCYL |
| | | N |
| Hymo_A_(partial) | 98 | CGETCLFIPCIFSVV |
| | | GCSCSSKVCYRN |
| Hymo_B_(partial) | 99 | CGETCVTGTCYTPGC |
| | | ACDWPVCKRD |
| Hyst_A_(partial) | 100 | CGETCIWGRCYSENI |
| | | GCHCGFGICTLN |
| Hyve_A_(partial) | 101 | CGETCLFIPCLTSVF |
| | | GCSCKNRGCYKI |
| Hyca_A_(partial) | 102 | CGETCVVDTRCYTKK |
| | | CSCAWPVCMRN |
| Hyde_A_(partial) | 103 | CVWIPCISAAIGCSC |
| | | KSKVCYRN |
| Hyen_A_(partial) | 104 | CGESCVYIPCTVTAL |
| | | LGCSCKDKVCYKN |
| Hyen_B_(partial) | 105 | CGETCKVTKRCSGQG |
| | | CSCLKGRSCYD |
| Hyep_A_(partial) | 106 | CGETCVVLPCFIVPG |
| | | CSCKSSVCYFN |
| Hyep_B_(partial) | 107 | CGETCIYIPCFTEAV |
| | | GCKCKDKVCYKN |
| tricyclon_B | 108 | GGTIFDCGESCFLGT |
| | | CYTKGCSCGEWKLCY |
| | | GEN |
| kalata_B8 | 109 | GSVLNCGETCLLGTC |
| | | YTTGCTCNKYRVCTK |
| | | D |
| cycloviolacin_H4 | 110 | GIPCAESCVWIPCTV |
| | | TALLGCSCSNNVCYN |
| cycloviolacin_O13 | 111 | GIPCGESCVWIPCIS |
| | | AAIGCSCKSKVCYRN |
| violacin_A | 112 | SAISCGETCFKFKCY |
| | | TPRCSCSYPVCK |
| cycloviolacin_O14 | 113 | GSIPACGESCFKGKC |
| | | YTPGCSCSKYPLCAK |
| | | N |
| cycloviolacin_O15 | 114 | GLVPCGETCFTGKCY |
| | | TPGCSCSYPICKKN |
| cycloviolacin_O16 | 115 | GLPCGETCFTGKCYT |
| | | PGCSCSYPICKKIN |
| cycloviolacin_O17 | 116 | GIPCGESCVWIPCIS |
| | | AAIGCSCKNKVCYRN |
| cycloviolacin_O18 | 117 | GIPCGESCVYIPCTV |
| | | TALAGCKCKSKVCYN |
| cycloviolacin_O19 | 118 | GTLPCGESCVWIPCI |
| | | SSVVGCSCKSKVCYK |
| | | D |
| cycloviolacin_O20 | 119 | GIPCGESCVWIPCLT |
| | | SAIGCSCKSKVCYRD |
| cycloviolacin_O21 | 120 | GLPVCGETCVTGSCY |
| | | TPGCTCSWPVCTRN |
| cycloviolacin_O22 | 121 | GLPICGETCVGGTCN |
| | | TPGCTCSWPVCTRN |
| cycloviolacin_O23 | 122 | GLPTCGETCFGGTCN |
| | | TPGCTCDSSWPICTH |
| | | N |
| cycloviolacin_O24 | 123 | GLPTCGETCFGGTCN |
| | | TPGCTCDPWPVCTHN |
| cycloviolacin_O25 | 124 | DIFCGETCAFIPCIT |
| | | HVPGTCSCKSKVCYF |
| | | N |
| [P20D,_V21K]- | 125 | GLPVCGETCVGGTCN |
| kalata_B1 | | TPGCTCSWDKCTRN |
| [W19K,_P20N,_V21K]- | 126 | GLPVCGETCVGGTCN |
| | | TPGCTCSKNKCTRN |
| kalata_B1 | | |
| [Glu(Me)]cyO2 | 127 | GIPCGXSCVWIPCIS |
| | | SAIGCSCKSKVCYRN |
| [Lys(Ac)]2cyO2 | 128 | GIPCGESCVWIPCIS |
| | | SAIGCSCXSXVCYRN |
| [Arg(CHD)]cyO2 | 129 | GIPCGESCVWIPCIS |
| | | SAIGCSCKSKVCYXN |
| ([Lys(Ac)]2 | 130 | GIPCGESCVWIPCIS |
| [Arg(CHD)]) | | SAIGCSCXSXVCYXN |
| cyO2 | | |
| kalata_B1_oia | 131 | GLPVCGETCVGGTCN |
| | | TPGCTCSWPVCTRN |
| kalata_B1_nfk | 132 | GLPVCGETCVGGTCN |
| | | TPGCTCSWPVCTRN |
| kalata_B2_nfk | 133 | GLPVCGETCFGGTCN |
| | | TPGCSCTWPICTRD |
| kalata_B2_kyn | 134 | GLPVCGETCFGGTCN |
| | | TPGCSCTWPICTRD |
| kalata_B9 | 135 | GSVFNCGETCVLGTC |
| | | YTPGCTCNTYRVCTK |
| | | D |
| kalata_B10 | 136 | GLPTCGETCFGGTCN |
| | | TPGCSCSSWPICTRD |
| kalata_B10_oia | 137 | GLPTCGETCFGGTCN |
| | | TPGCSCSSWPICTRD |
| kalata_B11 | 138 | GLPVCGETCFGGTCN |
| | | TPGCSCTDPICTRD |
| kalata_B12 | 139 | GSLCGDTCFVLGCND |
| | | SSCSCNYPICVKD |
| kalata_B13 | 140 | GLPVCGETCFGGTCN |
| | | TPGCACDPWPVCTRD |
| kalata_B14 | 141 | GLPVCGESCFGGTCN |
| | | TPGCACDPWPVCTRD |
| kalata_B15 | 142 | GLPVCGESCFGGSCY |
| | | TPGCSCTWPICTRD |
| kalata_B16 | 143 | GIPCAESCVYIPCTI |
| | | TALLGCKCQDKVCYD |
| kalata_B17 | 144 | GIPCAESCVYIPCTI |
| | | TALLGCKCKDQVCYN |
| Amrad_5 | 145 | CGETCVGGTCNTPGC |
| | | TCSWPVCRRKRRR |
| Amrad_9 | 146 | CGETCRRKRRRCNTP |
| | | GCTCSWPVCTRNGLP |
| | | V |
| Amrad_11 | 147 | CGETCVGGTCNTRRK |
| | | RRRGCTCSWPVCTRN |
| | | GLPV |
| Amrad_17 | 148 | CGETCVGGTCNTPGC |
| | | TCRRKRRRVCTRNGL |
| | | PV |
| Amrad_7 | 149 | CGETCVGGTCNTPGC |
| | | TCRRKRRRCTRNGLP |
| | | V |
| Amrad_8 | 150 | CGETCVGGTCRRKRR |
| | | RCTCSWPVCTRNGLP |
| | | V |
| kalata_B18 | 151 | GVPCAESCVYIPCIS |
| | | TVLGCSCSNQVCYRN |
| PS-1 | 152 | GFIPCGETCIWDKTC |
| | | HAAGCSCSVANICVR |
| | | N |
| CD-1 | 153 | GADGFCGESCYVIPC |
| | | ISYLVGCSCDTIEKV |
| | | CKRN |
| cycloviolacin_Y1 | 154 | GGTIFDCGETCFLGT |
| | | CYTPGCSCGNYGFCY |
| | | GTN |
| cycloviolacin_Y2 | 155 | GGTIFDCGESCFLGT |
| | | CYTAGCSCGNWGLCY |
| | | GTN |
| cycloviolacin_Y3 | 156 | GGTIFDCGETCFLGT |
| | | CYTAGCSCGNWGLCY |
| | | GTN |
| cycloviolacin_Y4 | 157 | GVPCGESCVFIPCIT |
| | | GVIGCSCSSNVCYLN |
| cycloviolacin_Y5 | 158 | GIPCAESCVWIPCTV |
| | | TALVGCSCSDKVCYN |
| vibi_A | 159 | GLPVCGETCFGGTCN |
| | | TPGCSCSYPICTRN |
| vibi_B | 160 | GLPVCGETCFGGTCN |
| | | TPGCTCSYPICTRN |
| vibi_C | 161 | GLPVCGETCAFGSCY |
| | | TPGCSCSWPVCTRN |
| vibi_D | 162 | GLPVCGETCFGGRCN |
| | | TPGCTCSYPICTRN |
| vibi_E | 163 | GIPCAESCVWIPCTV |
| | | TALIGCGCSNKVCYN |
| vibi_F | 164 | GTIPCGESCVFIPCL |
| | | TSALGCSCKSKVCYK |
| | | N |
| vibi_G | 165 | GTFPCGESCVFIPCL |
| | | TSAIGCSCKSKVCYK |
| | | N |
| vibi_H | 166 | GLLPCAESCVYIPCL |
| | | TTVIGCSCKSKVCYK |
| | | N |
| vibi_I | 167 | GIPCGESCVWIPCLT |
| | | STVGCSCKSKVCYRN |
| vibi_J | 168 | GTFPCGESCVWIPCI |
| | | SKVIGCACKSKVCYK |
| | | N |
| vibi_K | 169 | GIPCGESCVWIPCLT |
| | | SAVGCPCKSKVCYRN |
| Viba_2 | 170 | GIPCGESCVYLPCFT |
| | | APLGCSCSSKVCYRN |
| Viba_5 | 171 | GIPCGESCVWIPCLT |
| | | ATIGCSCKSKVCYRN |
| Viba_10 | 172 | GIPCAESCVYLPCVT |
| | | IVIGCSCKDKVCYN |
| Viba_12 | 173 | GIPCAESCVWIPCTV |
| | | TALLGCSCKDKVCYN |
| Viba_14 | 174 | GRLCGERCVIERTRA |
| | | WCRTVGCICSLHTLE |
| | | CVRN |
| Viba_17 | 175 | GLPVCGETCVGGTCN |
| | | TPGCGCSWPVCTRN |
| Viba_15 | 176 | GLPVCGETCVGGTCN |
| | | TPGCACSWPVCTRN |
| mram_1 | 177 | GSIPCGESCVYIPCI |
| | | SSLLGCSCKSKVCYK |
| | | N |
| mram_2 | 178 | GIPCAESCVYIPCLT |
| | | SAIGCSCKSKVCYRN |
| mram_3 | 179 | GIPCGESCVYLPCFT |
| | | TIIGCKCQGKVCYH |
| mram_4 | 180 | GSIPCGESCVFIPCI |
| | | SSVVGCSCKNKVCYK |
| | | N |
| mram_5 | 181 | GTIPCGESCVFIPCL |
| | | TSAIGCSCKSKVCYK |
| | | N |
| mram_6 | 182 | GSIPCGESCVYIPCI |
| | | SSLLGCSCESKVCYK |
| | | N |
| mram_7 | 183 | GSIPCGESCVFIPCI |
| | | SSIVGCSCKSKVCYK |
| | | N |
| mram_8 | 184 | GIPCGESCVFIPCLT |
| | | SAIGCSCKSKVCYRN |
| mram_9 | 185 | GVPCGESCVWIPCLT |
| | | SIVGCSCKNNVCTLN |
| mram_1 | 186 | GVIPCGESCVFIPCI |
| | | SSVLGCSCKNKVCYR |
| | | N |
| mram_11 | 187 | GHPTCGETCLLGTCY |
| | | TPGCTCKRPVCYKN |
| mram_12 | 188 | GSAILCGESCTLGEC |
| | | YTPGCTCSWPICTKN |
| mram_13 | 189 | GHPICGETCVGNKCY |
| | | TPGCTCTWPVCYRN |
| mram_14 | 190 | GSIPCGEGCVFIPCI |
| | | SSIVGCSCKSKVCYK |
| | | N |
| Viba_1 | 191 | GIPCGEGCVYLPCFT |
| | | APLGCSCSSKVCYRN |
| Viba_3 | 192 | GIPCGESCVWIPCLT |
| | | AAIGCSCSSKVCYRN |
| Viba_4 | 193 | GVPCGESCVWIPCLT |
| | | SAIGCSCKSSVCYRN |
| Viba_6 | 194 | GIPCGESCVLIPCIS |
| | | SVIGCSCKSKVCYRN |
| Viba_7 | 195 | GVIPCGESCVFIPCI |
| | | SSVIGCSCKSKVCYR |
| | | N |
| Viba_8 | 196 | GAGCIETCYTFPCIS |
| | | EMINCSCKNSRCQKN |
| Viba_9 | 197 | GIPCGESCVWIPCIS |
| | | SAIGCSCKNKVCYRK |
| Viba_11 | 198 | GIPCGESCVWIPCIS |
| | | GAIGCSCKSKVCYRN |
| Viba_13 | 199 | TIPCAESCVWIPCTV |
| | | TALLGCSCKDKVCYN |
| Viba_16 | 200 | GLPICGETCTLGTCY |
| | | TVGCTCSWPICTRN |
| [G1A]kalata_B1 | 201 | ALPVCGETCVGGTCN |
| | | TPGCTCSWPVCTRN |
| [L2A]kalata_B1 | 202 | GAPVCGETCVGGTCN |
| | | TPGCTCSWPVCTRN |
| [P3A]kalata_B1 | 203 | GLAVCGETCVGGTCN |
| | | TPGCTCSWPVCTRN |
| [V4A]kalata_B1 | 204 | GLPACGETCVGGTCN |
| | | TPGCTCSWPVCTRN |
| [G6A]kalata_B1 | 205 | GLPVCAETCVGGTCN |
| | | TPGCTCSWPVCTRN |
| [E7A]kalata_B1 | 206 | GLPVCGATCVGGTCN |
| | | TPGCTCSWPVCTRN |
| [T8A]kalata_B1 | 207 | GLPVCGEACVGGTCN |
| | | TPGCTCSWPVCTRN |
| [V10A]kalata_B1 | 208 | GLPVCGETCAGGTCN |
| | | TPGCTCSWPVCTRN |
| [G11A]kalata_B1 | 209 | GLPVCGETCVAGTCN |
| | | TPGCTCSWPVCTRN |
| [G12A]kalata_B1 | 210 | GLPVCGETCVGATCN |
| | | TPGCTCSWPVCTRN |
| [T13A]kalata_B1 | 211 | GLPVCGETCVGGACN |
| | | TPGCTCSWPVCTRN |
| [N15A]kalata_B1 | 212 | GLPVCGETCVGGTCA |
| | | TPGCTCSWPVCTRN |
| [T16A]kalata_B1 | 213 | GLPVCGETCVGGTCN |
| | | APGCTCSWPVCTRN |
| [P17A]kalata_B1 | 214 | GLPVCGETCVGGTCN |
| | | TAGCTCSWPVCTRN |
| [G18A]kalata_B1 | 215 | GLPVCGETCVGGTCN |
| | | TPACTCSWPVCTRN |
| [T20A]kalata_B1 | 216 | GLPVCGETCVGGTCN |
| | | TPGCACSWPVCTRN |
| [S22A]kalata_B1 | 217 | GLPVCGETCVGGTCN |
| | | TPGCTCAWPVCTRN |
| [W23A]kalata_B1 | 218 | GLPVCGETCVGGTCN |
| | | TPGCTCSAPVCTRN |
| [P24A]kalata_B1 | 219 | GLPVCGETCVGGTCN |
| | | TPGCTCSWAVCTRN |
| [V25A]kalata_B1 | 220 | GLPVCGETCVGGTCN |
| | | TPGCTCSWPACTRN |
| [T27A]kalata_B1 | 221 | GLPVCGETCVGGTCN |
| | | TPGCTCSWPVCARN |
| [R28A]kalata_B1 | 222 | GLPVCGETCVGGTCN |
| | | TPGCTCSWPVCTAN |
| [N29A]kalata_B1 | 223 | GLPVCGETCVGGTCN |
| | | TPGCTCSWPVCTRA |
| Cter_A | 224 | GVIPCGESCVFIPCI |
| | | STVIGCSCKNKVCYR |
| | | N |
| Cter_B | 225 | GVPCAESCVWIPCTV |
| | | TALLGCSCKDKVCYL |
| | | N |
| hcf-1_variant | 226 | GIPCGESCHIPCVTS |
| | | AIGCSCRNRSCMRN |
| Vpl-1 | 227 | GSQSCGESCVLIPCI |
| | | SGVIGCSCSSMICYF |
| | | N |
| Vpf-1 | 228 | GIPCGESCVFIPCLT |
| | | AAIGCSCRSKVCYRN |
| c031 | 229 | GLPVCGETCVGGTCN |
| | | TPGCSCSIPVCTRN |
| CO28 | 230 | GLPVCGETCVGGTCN |
| | | TPGCSCSWPVCFRD |
| c032 | 231 | GAPVCGETCFGGTCN |
| | | TPGCTCDPWPVCTND |
| cO33 | 232 | GLPVCGETCVGGTCN |
| | | TPYCTCSWPVCTRD |
| cO34 | 233 | GLPVCGETCVGGTCN |
| | | TEYCTCSWPVCTRD |
| c035 | 234 | GLPVCGETCVGGTCN |
| | | TPYCFCSWPVCTRD |
| c029 | 235 | GIPCGESCVWIPCIS |
| | | GAIGCSCKSKVCYKN |
| CO30 | 236 | GIPCGESCVWIPCIS |
| | | SAIGCSCKNKVCFKN |
| c026 | 237 | GSIPACGESCFRGKC |
| | | YTPGCSCSKYPLCAK |
| | | D |
| CO27 | 238 | GSIPACGESCFKGWC |
| | | YTPGCSCSKYPLCAK |
| | | D |
| Globa_F | 239 | GSFPCGESCVFIPCI |
| | | SAIAGCSCKNKVCYK |
| | | N |
| Globa_A | 240 | GIPCGESCVFIPCIT |
| | | AAIGCSCKTKVCYRN |
| Globa_B | 241 | GVIPCGESCVFIPCI |
| | | SAVLGCSCKSKVCYR |
| | | N |
| Globa_D | 242 | GIPCGETCVFMPCIS |
| | | GPMGCSCKHMVCYRN |
| Globa_G | 243 | GVIPCGESCVFIPCI |
| | | SSVLGCSCKNKVCYR |
| | | N |
| Globa_E | 244 | GSAFGCGETCVKGKC |
| | | NTPGCVCSWPVCKKN |
| Globa_C | 245 | APCGESCVFIPCISA |
| | | VLGCSCKSKVCYRN |
| Glopa_D | 246 | GVPCGESCVWVPCTV |
| | | TALMGCSCVREVCRK |
| | | D |
| Glopa_E | 247 | GIPCAESCVWIPCTV |
| | | TKMLGCSCKDKVCYN |
| Glopa_A | 248 | GGSIPCIETCVWTGC |
| | | FLVPGCSCKSDKKCY |
| | | LN |
| Glopa_B | 249 | GGSVPCIETCVWTGC |
| | | FLVPGCSCKSDKKCY |
| | | LN |
| Glopa_C | 250 | GDIPLCGETCFEGGN |
| | | CRIPGCTCVWPFCSK |
| | | N |
| Co36 | 251 | GLPTCGETCFGGTCN |
| | | TPGCTCDPFPVCTHD |
| cycloviolacin_T1 | 252 | GIPVCGETCVGGTCN |
| | | TPGCSCSWPVCTRN |
| cycloviolacin_T2 | 253 | GLPICGETCVGGTCN |
| | | TPGCSCSWPVCTRN |
| psyle_A | 254 | GIACGESCVFLGCFI |
| | | PGCSCKSKVCYFN |
| psyle_B | 255 | GIPCGETCVAFGCWI |
| | | PGCSCKDKLCYYD |
| psyle_C | 256 | KLCGETCFKFKCYTP |
| | | GCSCSYFPCK |
| psyle_D | 257 | GIPCGESCVFIPCTV |
| | | TALLGCSCQNKVCYR |
| | | D |
| psyle_E | 258 | GVIPCGESCVFIPCI |
| | | SSVLGCSCKNKVCYR |
| | | D |
| psyle_F | 259 | GVIPCGESCVFIPCI |
| | | TAAVGCSCKNKVCYR |
| | | D |
| vaby_A | 260 | GLPVCGETCAGGTCN |
| | | TPGCSCSWPICTRN |
| vaby_B | 261 | GLPVCGETCAGGTCN |
| | | TPGCSCTWPICTRN |
| vaby_C | 262 | GLPVCGETCAGGRCN |
| | | TPGCSCSWPVCTRN |
| vaby_D | 263 | GLPVCGETCFGGTCN |
| | | TPGCTCDPWPVCTRN |
| vaby_E | 264 | GLPVCGETCFGGTCN |
| | | TPGCSCDPWPVCTRN |
| Oak6_cyclotide_2 | 265 | GLPICGETCFGGTCN |
| | | TPGCICDPWPVCTRD |
| Oak7_cyclotide | 266 | GSHCGETCFFFGCYK |
| | | PGCSCDELRQCYKN |
| Oak8_cyclotide | 267 | GVPCGESCVFIPCLT |
| | | AVVGCSCSNKVCYLN |
| Oak6_cyclotide_1 | 268 | GLPVCGETCFGGTCN |
| | | TPGCACDPWPVCTRN |
| Cter_C | 269 | GVPCAESCVWIPCTV |
| | | TALLGCSCKDKVCYL |
| | | D |
| Cter_D | 270 | GIPCAESCVWIPCTV |
| | | TALLGCSCKDKVCYL |
| | | N |
| Cter_E | 271 | GIPCAESCVWIPCTV |
| | | TALLGCSCKDKVCYL |
| | | D |
| Cter_F | 272 | GIPCGESCVFIPCIS |
| | | SVVGCSCKSKVCYLD |
| Cter_G | 273 | GLPCGESCVFIPCIT |
| | | TVVGCSCKNKVCYNN |
| Cter_H | 274 | GLPCGESCVFIPCIT |
| | | TVVGCSCKNKVCYND |
| Cter_I | 275 | GTVPCGESCVFIPCI |
| | | TGIAGCSCKNKVCYI |
| | | N |
| Cter_J | 276 | GTVPCGESCVFIPCI |
| | | TGIAGCSCKNKVCYI |
| | | D |
| Cter_K | 277 | HEPCGESCVFIPCIT |
| | | TVVGCSCKNKVCYN |
| Cter_L | 278 | HEPCGESCVFIPCIT |
| | | TVVGCSCKNKVCYD |
| Cter_M | 279 | GLPTCGETCTLGTCY |
| | | VPDCSCSWPICMKN |
| Cter_N | 280 | GSAFCGETCVLGTCY |
| | | TPDCSCTALVCLKN |
| Cter_O | 281 | GIPCGESCVFIPCIT |
| | | GIAGCSCKSKVCYRN |
| Cter_P | 282 | GIPCGESCVFIPCIT |
| | | AAIGCSCKSKVCYRN |
| Cter_Q | 283 | GIPCGESCVFIPCIS |
| | | TVIGCSCKNKVCYRN |
| Cter_R | 284 | GIPCGESCVFIPCTV |
| | | TALLGCSCKDKVCYK |
| | | N |
| vitri_B | 285 | GVPICGESCVGGTCN |
| | | TPGCSCSWPVCTTN |
| vitri_C | 286 | GLPICGETCVGGTCN |
| | | TPGCFCTWPVCTRN |
| vitri_D | 287 | GLPVCGETCFTGSCY |
| | | TPGCSCNWPVCNRN |
| vitri_E | 288 | GLPVCGETCVGGTCN |
| | | TPGCSCSWPVCFRN |
| vitri_F | 289 | GLTPCGESCVWIPCI |
| | | SSVVGCACKSKVCYK |
| | | D |
| hedyotide_B1 | 290 | GTRCGETCFVLPCWS |
| | | AKFGCYCQKGFCYRN |

In one embodiment, the cyclotide incorporates one or more unnatural amino acids. “Unnatural amino acids” are non-proteinogenic amino acids that either occur naturally or are chemically synthesized. While unnatural amino acids are not on the standard 20-amino acid list, they can be incorporated into a protein sequence. Non-limiting examples of unnatural amino acids include L-2,3-diaminopropionic acid, DL-2,3-diaminopropionic acid, 2,4-diaminobutyric acid, p-methyxyphenylalanine, p-azidophenylalanine, L-(7-hydroxycoumarin-4-yl)ethylglycine, acetyl-2-naphthyl alanine, 2-naphthyl alanine, 3-pyridyl alanine, 4-chloro phenyl alanine, alloisoleucine, Z-alloisoleucine dcha salt, allothreonine, 4-iodo-phenylalanine, L-benzothienyl-D-alanine OH.

In some aspects the cyclotide comprises at least an unnatural amino acid residue but retains six cysteine residues that form three disulfide bonds in a cyclized cyclotide. In one aspect, the unnatural amino acid comprises one or more selected from L-2,3-diaminopropionic acid (L-Dap), p-methyxyphenylalanine, p-azidophenylalanine or L-(7-hydroxycoumarin-4-yl)ethylglycine.

In some embodiments, the cyclotide backbone is comprised of, or alternatively consists essentially a peptide from Momordica spp plants (see FIGS. 11A and 11B). In one aspect, the cyclotide backbone is MCoTI-I. The sequence of MCoTI-I is described FIG. 1. In one aspect, MCoTI-I comprises the sequence GGVCPKILQRCRRDSDCPGACICRGNGYCGSGSD (SEQ ID NO: 1), or an equivalent thereof. In another aspect, the sequence comprises GGBCPKILQRCRRDSDCPGACICRGAGYCGSGSD (SEQ ID NO: 2), or an equivalent thereof. In some aspects, two residues are removed from the carboxy terminal end so that the sequence of MCoTI-I comprises or consists essentially of GGBCPKILQRCRRDSDCPGACICRGAGYCGSG (SEQ ID NO: 3) or GGVCPKILQRCRRDSDCPGACICRGNGYCGSG (SEQ ID NO: 4). In further aspect, residue B in the above sequences represents asparagine or aspartic acid.

In one embodiment, the PG-1 polypeptide is grafted into any one of loops 1 to 6 of the cyclotide backbone. In another embodiment, the PG-1 polypeptide is grafted into loop 6 of the cyclotide backbone. The cyclotide comprises a molecular framework comprising a sequence of amino acids forming a cyclic backbone wherein the cyclic backbone comprises sufficient disulfide bonds to confer knotted topology on the molecular framework or part thereof. Examples of cyclic backbone polypeptides are now in the art and described herein.

The antimicrobials as described herein can further comprising a label or purification marker and/or a carrier, such as a pharmaceutically acceptable carrier.

Also provided is a plurality of antimicrobials as described herein that may be the same or different from each other. These can further comprising a carrier such as a pharmaceutically acceptable carrier.

In another aspect, the carrier further comprises an additional antibiotic or antimicrobial.

Yet further provided are isolated polynucleotides encoding the antimicrobials as described herein as well as a complement of each. In one aspect, the isolated polynucleotide or complement further comprises a label or a purification marker and can be combined with a carrier, such as a pharmaceutically acceptable carrier. The polynucleotides can be operatively linked to elements for replication or expression, such as promoters and enhancers. Means to create such recombinant polynucleotides are known in the art. The polynucleotides can be contained with a vector such as a plasmid or viral vector for recombinant duplication or expression. The expressed antimicrobial can be purified from the cell or its environment.

Also provided herein is a prokaryotic or eukaryotic host cell comprising one or more of the antimicrobial, polynucleotide, or vector as described herein. The host cells can be used to recombinantly express the polynucleotide encoding the antimicrobial by growing the isolated host cell comprising a polynucleotide encoding such under conditions that express the polynucleotide. In a further aspect, the antimicrobial is purified from the host cell or its environment such as the cell culture conditions.

### Compositions

Compositions are further provided. The compositions comprise a carrier and one or more of an antimicrobials of the disclosure or a polynucleotide encoding same, a vector containing the polynucleotide or a host cell containing one or more of the antimicrobial, the polynucleotide or vector. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include one or more of an antimicrobial, an isolated polynucleotide of the disclosure, a vector of the disclosure, an isolated host cell of the disclosure, formulated with one or more pharmaceutically acceptable auxiliary substances.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The pharmaceutical compositions can be formulated into preparations for administration in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other anticancer agents. For intravenous administration, suitable carriers include physiological saline, or phosphate buffered saline (PBS). In all cases, a composition for parenteral administration must be sterile and should be fluid to the extent that easy syringeability exists.

Aerosol formulations provided by the disclosure can be administered via inhalation and can be propellant or non-propellant based. For example, embodiments of the pharmaceutical formulations of the disclosure comprise a peptide of the disclosure formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself (e.g. by an elastic bladder)).

Suppositories of the disclosure can be prepared by mixing a compound of the disclosure with any of a variety of bases such as emulsifying bases or water soluble bases. Embodiments of this pharmaceutical formulation of a compound of the disclosure can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds of the disclosure. Similarly, unit dosage forms for injection or intravenous administration may comprise a compound of the disclosure in a composition as a solution in sterile water, normal saline or another pharmaceutically acceptable carrier.

Embodiments of the pharmaceutical formulations of the disclosure include those in which one or more of an isolated polypeptide of the disclosure, an isolated polynucleotide of the disclosure, a vector of the disclosure, an isolated host cell of the disclosure, or an antibody of the disclosure is formulated in an injectable composition. Injectable pharmaceutical formulations of the disclosure are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the disclosure.

In an embodiment, one or more of an isolated polypeptide of the disclosure, an isolated polynucleotide of the disclosure, a gene delivery vehicle or vector of the disclosure, or an isolated host cell of the disclosure is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound of the disclosure can be accomplished using any of a variety of refillable, pump systems. Pumps provide consistent, controlled release over time. In some embodiments, a compound of the disclosure is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a peptide of the disclosure are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. After administration, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the antimicrobial (as well as combination compositions) is delivered in a controlled release system. For example, the antimicrobial of the disclosure may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the lung, requiring only a fraction of the systemic dose.

In another embodiment, the compositions of the present disclosure (as well as combination compositions separately or together) include those formed by impregnation of a peptide described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The compositions can comprise an additional antimicrobial, antibiotic or vaccine formulation for use as described herein. Non-limiting examples include piperacillin, ceftazidime, sulfonamide, a β-lactam antibiotic, tobramycin, colisin, trimethoprim, sulfamethoxazole, clarithromycin, glutamate, ampicillin, amoxicillin, clavulanate or cefdinir. In one aspect, two or more are provided, glutamate and tobramycin, glutamate and colisin, trimethoprim and sulfamethoxazole, trimethoprim and clarithromycin, amoxicillin and clavulanate, or trimethoprim and sulfamethoxazole.

### Methods of Use

The antimicrobials of this disclosure are useful in a variety of in vitro and in vivo methods. In one aspect, the antimicrobials are administered to a subject in need thereof to inhibit the growth of a microorganism or treat an infection by the microorganism in a subject in need thereof. In another aspect, the antimicrobial is provided to inhibit the growth of a microorganism or a cell containing same by contacting the cell or organism with an effective amount of the antimicrobial, the plurality, the composition, polynucleotide or cell as described herein.

The disclosed above methods comprising contacting the cell or microorganism with an effective amount of one or more of: the antimicrobials as described herein, the pluralities, the compositions, the isolated polynucleotide described herein or the host cell described herein. The inhibition of the growth or the treatment of an infection can be detected by methods known in the art and described herein. The contacting of the cell or tissue may be in vitro in tissue culture or in vivo in a subject.

The compositions can be administered to an animal or mammal by a treating veterinarian or to a human patient by a treating physician.

In one aspect, provided is a method for one or more of the following: inhibiting, preventing or treating a microbial infection that produces a biofilm in a subject, treating a condition characterized by the formation of a biofilm in a subject. The method comprises, or consists essentially of, or yet further consists of administering to the subject one or more of: a composition as disclosed herein, an antimicrobial as disclosed herein, a polynucleotide as disclosed herein, a vector as disclosed herein, or a host cell as disclosed herein. In some aspects relating to any method(s) as disclosed herein, optionally comprising an administration step an additional antimicrobial or biofilm disrupting agent. In some aspects, the condition characterized by the formation of a biofilm or is associated with a biofilm and is selected from the group consisting of: chronic non-healing wounds, Burkholderia, venous ulcers, diabetic foot ulcers, ear infections, sinus infections, urinary tract infections, gastrointestinal tract ailments, pulmonary infections, respiratory tract infections, cystic fibrosis, chronic obstructive pulmonary disease, catheter-associated infections, indwelling devices associated infections, infections associated with implanted prostheses, osteomyelitis, cellulitis, abscesses, and periodontal disease.

When practiced in vivo in non-human animal such as a chinchilla, the method provides a pre-clinical screen to identify agents that can be used alone or in combination with other agents to treat infections and in one aspect, biofilm associated diseases. A sample from the patient or subject can be isolated and used in an in vitro assay to determine inhibitory effect.

The compositions can be combined with other antimicrobials antibiotics or vaccine formulations. Non-limiting examples include piperacillin, ceftazidime, sulfonamide, a β-lactam antibiotic, tobramycin, colisin, trimethoprim, sulfamethoxazole, clarithromycin, glutamate, ampicillin, amoxicillin, clavulanate or cefdinir. In one aspect, two or more are provided, glutamate and tobramycin, glutamate and colisin, trimethoprim and sulfamethoxazole, trimethoprim and clarithromycin, amoxicillin and clavulanate, or trimethoprim and sulfamethoxazole.

A non-limiting example of a vaccine component such as a surface antigen, e.g., an OMP P5, rsPilA, OMP 26, OMP P2, or Type IV Pilin protein (see Jurcisek and Bakaletz (2007) J. Bacteriology 189(10):3868-3875; Murphy, T. F. et al. (2009) The Pediatric Infectious Disease Journal 28:S121-S126).

The agents and compositions disclosed herein can be concurrently or sequentially administered with other antimicrobial agents and/or surface antigens. In one particular aspect, administration is locally to the site of the infection by direct injection or by inhalation for example. Other non-limiting examples of administration include by one or more method comprising transdermally, urethrally, sublingually, rectally, vaginally, ocularly, subcutaneous, intramuscularly, intraperitoneally, intranasally, by inhalation or orally.

Microbial infections and disease that can be treated by the methods disclosed herein include infection by a gram-positive or gram-negative organism that produces a biofilm, e.g., Streptococcus agalactiae, Neisseria meningitidis, Treponemes, denticola, pallidum, Burkholderia cepacia, or Burkholderia pseudomallei. In one aspect, the microbial infection is one or more of Haemophilus influenzae (nontypeable), Moraxella catarrhalis, Streptococcus pneumoniae, Streptococcus pyogenes, Pseudomonas aeruginosa, Mycobacterium tuberculosis. These microbial infections may be present in the upper, mid and lower airway (otitis, sinusitis, bronchitis but also exacerbations of chronic obstructive pulmonary disease (COPD), chronic cough, complications of and/or primary cause of cystic fibrosis (CF) and community acquired pneumonia (CAP). Thus, by practicing the in vivo methods disclosed herein, these diseases and complications from these infections can also be prevented or treated.

Infections might also occur in the oral cavity (caries, periodontitis) and caused by Streptococcus mutans, Porphyromonas gingivalis, Aggregatibacter actinomvctemcomitans. Infections might also be localized to the skin (abscesses, ‘staph’ infections, impetigo, secondary infection of burns, Lyme disease) and caused by Staphylococcus aureus, Staphylococcus epidermidis, Pseudomonas aeruginosa and Borrelia burdorferi. Infections of the urinary tract (UTI) can also be treated and are typically caused by Escherichia coli. Infections of the gastrointestinal tract (GI) (diarrhea, cholera, gall stones, gastric ulcers) are typically caused by Salmonella enterica serovar, Vibrio cholerae and Helicobacter pylori. Infections of the genital tract include and are typically caused by Neisseria gonorrhoeae. Infections can be of the bladder or of an indwelling device caused by Enterococcus faecalis. Infections associated with implanted prosthetic devices, such as artificial hip or knee replacements, or dental implants, or medical devices such as pumps, catheters, stents, or monitoring systems, typically caused by a variety of bacteria, can be treated by the methods disclosed herein. These devices can be coated or conjugated to an agent as described herein. Thus, by practicing the in vivo methods disclosed herein, these diseases and complications from these infections can also be prevented or treated.

Infections caused by Streptococcus agalactiae can also be treated by the methods disclosed herein and it is the major cause of bacterial septicemia in newborns. Infections caused by Neisseria meningitidis which can cause meningitis can also be treated.

Thus, routes of administration applicable to the methods disclosed herein include intranasal, intramuscular, urethrally, intratracheal, subcutaneous, intradermal, transdermal, topical application, intravenous, rectal, nasal, oral, inhalation, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery include systemic or localized routes. In general, routes of administration suitable for the methods disclosed herein include, but are not limited to, direct injection, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

The agents disclosed herein can also be delivered to the subject by enteral administration. Enteral routes of administration include, but are not limited to, oral and rectal (e.g., using a suppository) delivery.

Methods of administration of the active through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods disclosed herein, the interfering agent will be administered by inhalation, injection or orally on a continuous, daily basis, at least once per day (QD), and in various embodiments two (BID), three (TID), or even four times a day. Typically, the therapeutically effective daily dose will be at least about 1 mg, or at least about 10 mg, or at least about 100 mg, or about 200 to about 500 mg, and sometimes, depending on the compound, up to as much as about 1 g to about 2.5 g.

Dosing of can be accomplished in accordance with the methods disclosed herein using capsules, tablets, oral suspension, suspension for intra-muscular injection, suspension for intravenous infusion, get or cream for topical application, or suspension for intra-articular injection.

Dosage, toxicity and therapeutic efficacy of compositions described herein can be determined by standard pharmaceutical procedures in cell cultures or experimental animals, for example, to determine the LD50 (the dose lethal to 50% of the population) and the ED50 (the dose therapeutically effective in 50% of the population). The dose ratio between toxic and therapeutic effects is the therapeutic indexed it can be expressed as the ratio LD50/ED50. In certain embodiments, compositions exhibit high therapeutic indices. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies (in certain embodiments, within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, an effective amount of a composition sufficient for achieving a therapeutic or prophylactic effect, ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more “booster” doses. Booster doses can be provided a day, two days, three days, a week, two weeks, three weeks, one, two, three, six or twelve months after an initial dose. In some embodiments, a booster dose is administered after an evaluation of the subject's response to prior administrations.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

Having described the general concepts of this invention, the following illustrative examples are provided.

### Experimental Discussion

In order to produce a cyclotide with PG-1 antimicrobial activity, Applicant employed the naturally-occurring cyclotide MCoTI-I as molecular framework (FIG. 1). MCoTI-cyclotides are potent trypsin inhibitors isolated from the seeds of Momordica cochinchinensis[22] and show very low toxicity in human cells,[19b, 20] and therefore represent a desirable molecular scaffold for engineering new cyclotides with minimal toxicity and novel biological activities.[20, 23]

According to the solution structure of PG-1,[7b] its N- and C-termini are very close in space although the N-terminus is slightly more extended (FIG. 1). Therefore, a modified version of PG-1, where the N-terminal Arg residue was moved to the C-terminal position of the PG-1 sequence, was grafted into loop 6 of the cyclotide MCoTI-I (cyclotide MCo-PG2, FIG. 1) although other cyclotide backbones and/or loops can be employed. Applicant also added an extra disulfide to the grafted PG-1-derived sequence to further stabilize the grafted β-hairpin structure. This was accomplished by replacing both residues Arg4 and Gly17 in the original PG-1 sequence with Cys residues (MCo-PG3, FIG. 1). Similar modifications can be made to other loops and/or backbone structures. Two more cyclotides were also designed with longer and shorter versions of the PG-1-based grafted sequence to explore the effect of the distance of the grafted sequence from the cyclotide and to minimize the size of the PG-1-derived graft (FIG. 1). The elongated version (MCo-PG4) was obtained by adding two extra Gly residues to the N- and C-terminal positions of the modified PG-1 sequence. The shorten version (MCo-PG5) removed the N- and C-terminal Gly and Arg residues from the modified PG-1 sequence, respectively. These analogs were designed to explore the effect of the distance of the grafted sequence from the cyclotide and to minimize the size of the PG-1-derived graft. The different sequences were grafted onto loop 6 of cyclotide MCoTI-I by replacing residue Asp34 (FIG. 1) as this loop has been shown to be less rigid in solution [24] and quite tolerant to sequence grafting of relatively long peptide sequences [20, 23d, 23e, 25] although other loops can be similarly modified.

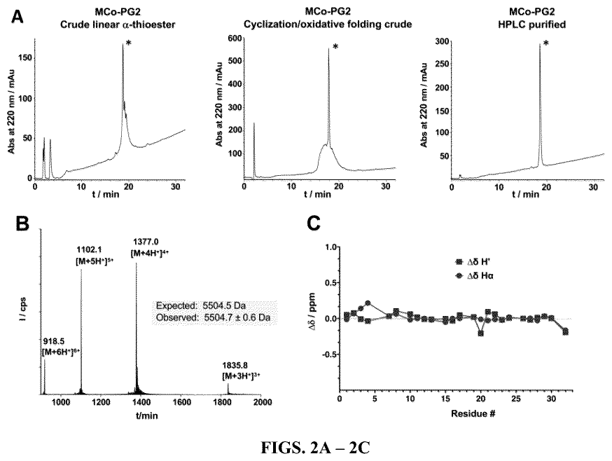

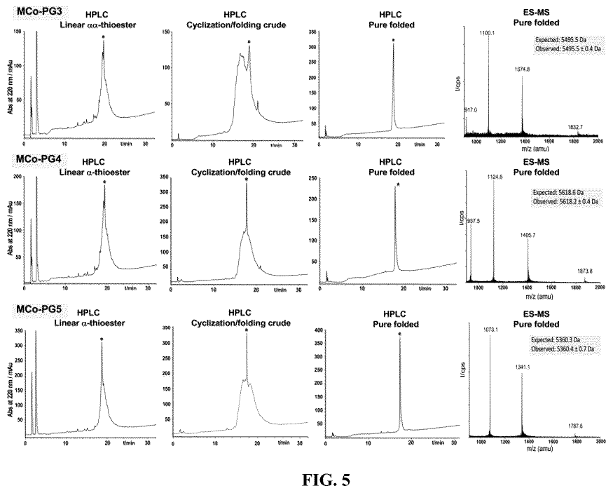

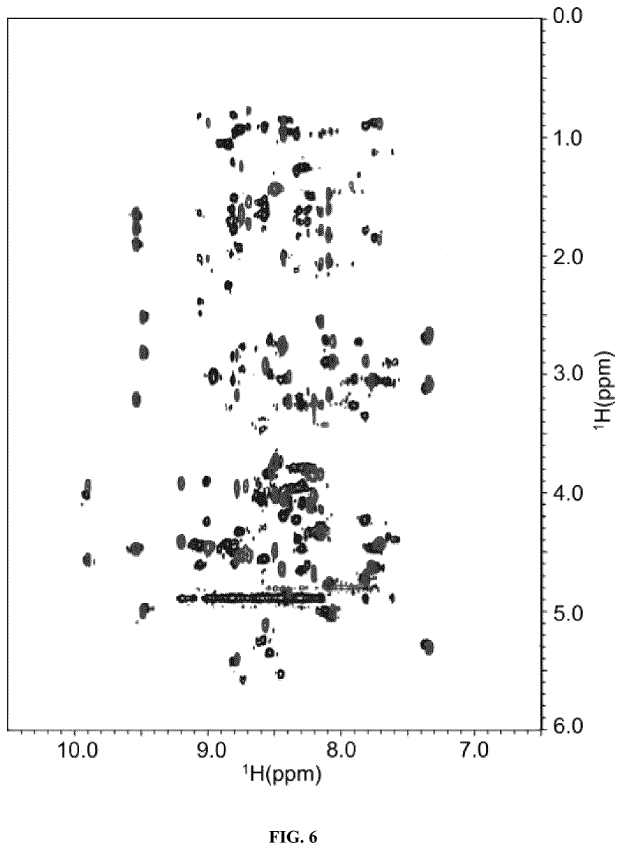

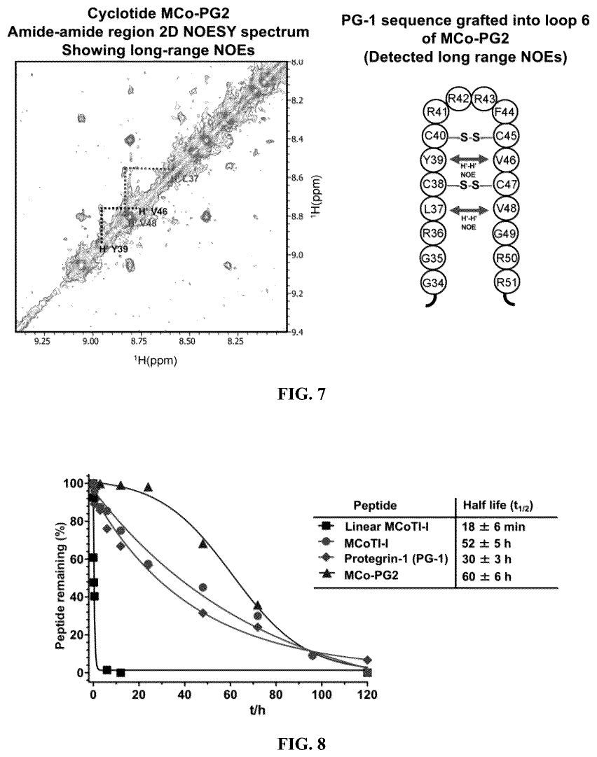

All grafted MCo-PG cyclotides were chemically synthesized on a sulfonamide resin using an Fmoc-based solid-phase peptide synthesis protocol [19b] The corresponding fully deprotected linear peptide α-thioesters were obtained by alkylation of the sulfonamide linker followed by thiolytic cleavage of the alkylated sulfonamide linker and acidolytic deprotection of the side-chain protecting groups. Cyclization and oxidative folding were accomplished in a one-pot reaction under thermodynamic control using aqueous buffer at pH 7.4 in the presence of 1 mM reduced glutathione (GSH). In all the cases the cyclization/folding reactions were complete in 72-96 h (FIG. 2A and FIG. 5). The yields for the cyclization/folding reactions ranged from 16% (MCo-PG3) to 40% (MCo-PG2) (Table 3). All folded cyclotides were purified by reverse-phase HPLC and characterized by ES-MS (FIG. 1B and FIG. 5, Table 3). In addition, cyclotide MCo-PG2 was also characterized by homonuclear NMR spectroscopy. The chemical shift A6 values for most of the backbone protons for the common part shared with the parent cyclotide MCoTI-I were smaller than 0.1 ppm indicating that MCo-PG2 adopts a native cyclotide fold (FIG. 2C and FIG. 6, Table 4). Analysis of the through space nuclear Overhauser effect, NOE, connectivities in the 2D ¹H-¹H NOESY spectrum of cyclotide MCo-PG2 revealed long-range NOEs between the backbone H′ protons from residues Leu[37] and Val[48], and residues Tyr[39] and Val[46] in the protegrin-derived graft of cyclotide MCo-PG2; these NOEs are also present in PG-1[7b] and are characteristic of a native 0-hairpin fold (FIG. 7).

Applicant then tested the broad-spectrum antimicrobial activity of the different PG-1-grafted cyclotides against different strains of four ESKAPE pathogens, P. aeruginosa, S. aureus, K. pneumoniae, and E. coli (Table 1). The naturally occurring cyclotide MCoTI-I and the porcine protegrin PG-1 were used as negative and positive controls, respectively. The minimum inhibitory concentration (MIC) values for the different peptides were determined by broth microdilution assay using a cation-adjusted Mueller-Hinton broth (CAMHB).[26] This growth medium contains 128 mM NaCl supplemented with calcium and magnesium salts providing very similar ionic strength to those found under physiological conditions. As expected, protegrin PG-1 exhibited potent and strong activity against Gram-negative and Gram-positive bacteria, with MIC values ranging from 0.03 μM (E. coli DS377) to 0.4 μM (S. aureus USA300 and HH35, both methicillin resistant strains; and K. pneumoniae BAA1705 and K6) (Table 1). This result is an agreement with published data for this protegrin.[26] Interestingly, all PG-1-grafted MCoTI-based cyclotides showed antibacterial activity against P. aeruginosa, with MIC values from 25 μM for the less active cyclotide (MCo-PG3) to 1.6 μM for the most active cyclotides (MCo-PG2 and MCo-PG4) (Table 1). Cyclotide MCo-PG3 also showed little activity against S. aureus, K. pneumoniae and E. coli, with MIC values in all the cases above 25 μM, indicating that addition of an extra-disulfide bond to the grafted peptide significantly reduced its antimicrobial activity. Shortening the grafted PG-1-derived sequence also had a detrimental effect on the antimicrobial activity of cyclotide MCo-PG5 although the effect was not as pronounced as the observed for cyclotide MCo-PG3. Elongation of the grafted sequence by adding extra Gly residues had very little impact on the antimicrobial activity, with cyclotides MCo-PG2 and MCo-PG3 showing similar the same antibacterial activity. As shown in Table 1, MCo-PG2 was slightly more active than MCo-PG4 against P. aeruginosa, S. aureus and E. coli, but slightly less active against K. pneumoniae. As expected, the naturally-occurring cyclotide MCoTI-I did not show any antibacterial activity in this assay up to a concentration of 200 μM (Table 1), indicating that the antimicrobial activity of PG-1 grafted cyclotides was specific and comes from the grafted sequence.

Based on the superior spectrum of activity of cyclotide MCo-PG2 against three of the four ESKAPE pathogens tested in this study, and in particular P. aeruginosa and S. aureus, which are two ESKAPE pathogens that commonly infect the airways of patients with cystic fibrosis, the antimicrobial activity of cyclotide MCo-PG2 was tested against 20 different clinical isolates of P. aeruginosa and S. aureus. These strains were collected from patients suffering from cystic fibrosis at the Keck Medical Center, University of Southern California (Table 2). Remarkably, MCo-PG2 retained its antimicrobial activity against P. aeruginosa and S. aureus clinical isolates, with MIC values ranging from 0.4 μM to 12.5 μM (Table 2). The median MIC (MIC₅₀) and MIC 90% (MIC₉₀) values for the P. aeruginosa population (n=20) were 1.5 μM while and 3.1 μM, respectively. For the S. aureus isolates (n=20), the MIC₅₀ and MIC₉₀ were 6.25 μM and 12.5 μM, respectively, indicating that MCo-PG2 shows four times better antimicrobial activity (MIC₉₀ values) against P. aeruginosa than to S. aureus stains. In comparison to protegrin PG-1, cyclotide MCo-PG2 was around four and ten times less active (MIC₉₀ values) against P. aeruginosa and S. aureus than the natural protegrin peptide (Table 2). These results were extremely encouraging indicating cyclotide MCo-PG2 was able to maintain good MIC values against pathogenic clinical isolates. It is important to remark that 30% of the P. aeruginosa clinical isolates were multidrug resistant strains (MDR), while 100% of the S. aureus clinical strains were methicillin-resistant, hence further highlighting the significance of MCo-PG2 MIC values against these pathogens.

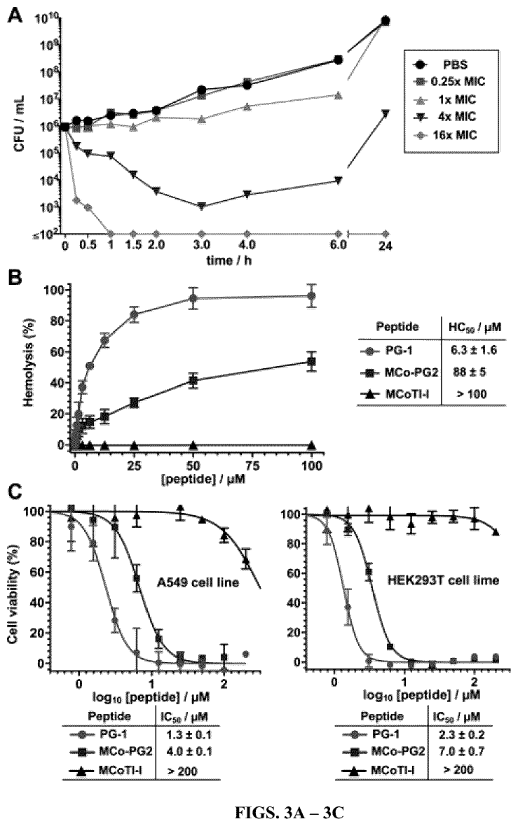

A time-kill kinetic assay was run to establish the bactericidal activity of cyclotide MCo-PG2 against P. aeruginosa PAO1 (FIG. 3A). This was accomplished by using different MCo-PG2 concentrations ranging from 0.25×MIC to 16×MIC values. The results indicated a rapid and concentration dependent killing kinetics against P. aeruginosa PAO1 by MCo-PG2 with greater than 3 log₁₀ CFU/mL bactericidal activity at concentrations of 4 times the MIC value. It is important to highlight that by using 16 times the MIC value of MCo-PG2 no regrowth of P. aeruginosa after 24 h was observed (FIG. 3A).

Applicant also evaluated the hemolytic activity of cyclotide MCo-PG2. As shown in FIG. 3B, MCo-PG2 exhibited a significantly lower hemolytic activity (HC₅₀=88±5 μM) than that of protegrin PG-1 (HC₅₀=6.3±1.6 μM). As expected, the control cyclotide MCoTI-I did not have any hemolytic activity up to a concentration of 100 μM. The membranolytic selectivity index (HC₅₀/MIC) is often used as an indicator of the therapeutic potential of a peptide-based antibiotic.[27] The HC₅₀/MIC₅₀ values for MCo-PG2 and PG-1 against P. aeruginosa clinical isolates were around 60 and 32, respectively. The HC₅₀/MIC₅₀ values for S. aureus clinical strains were found to be similar for PG-1 and MCo-PG2 with value around 15. These results indicate that cyclotide MCo-PG2 has greater therapeutic potential than PG-1 against P. aeruginosa, while showing similar therapeutic potential against S. aureus.

The cytotoxicity profile of cyclotide MCo-PG2 was also studied using two types of human epithelial cells: HEK293T (transformed kidney epithelial cells) and A549 (lung carcinoma). As shown in FIG. 3C, the cyclotide MCo-PG2 was about three times less toxic than PG-1. As previously reported, [20] the control cyclotide MCoTI-I did not present any cytotoxicity in human cells up to 100 μM.

The biological stability of cyclotide MCo-PG2 was explored and compared to that of the empty scaffold (MCoTI-I) and protegrin PG-1 (FIG. 8). This was accomplished by incubating the corresponding peptides in human serum at 37° C. The quantitative analysis of undigested polypeptides was performed using liquid chromatography coupled with tandem mass spectrometry (LC-MS/MS). MCoTI-cyclotides present a very rigid structure,[24] which makes them extremely stable to proteolytic degradation. Remarkably, cyclotide MCo-PG2 showed slightly greater stability in human serum (t_{1/2}=60±6 h) than the parent cyclotide MCoTI-I (t_{1/2}=52±5 h, FIG. 8). More importantly, cyclotide MCo-PG2 displayed minimal degradation within the first 24 h of the serum stability assay, while 40% of cyclotide MCoTI-I was degraded during the first 24 h of the assay (FIG. 8). In contrast, protegrin PG-1 was degraded significantly faster than MCo-PG2 (t_{1/2}=30±3 h) also showing significant degradation after 24 h of incubation with serum. A linearized, reduced and alkylated version of MCoTI-I was used as positive control and as expected was rapidly degraded (t_{1/2}=18±6 min). These results highlight the importance of the circular Cys-knot topology for proteolytic stability.

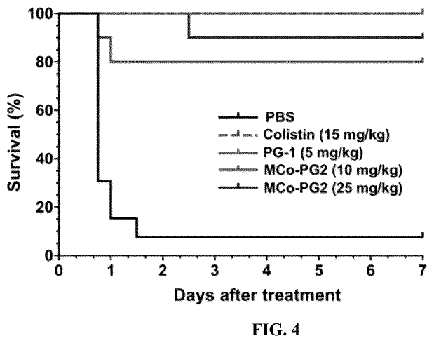

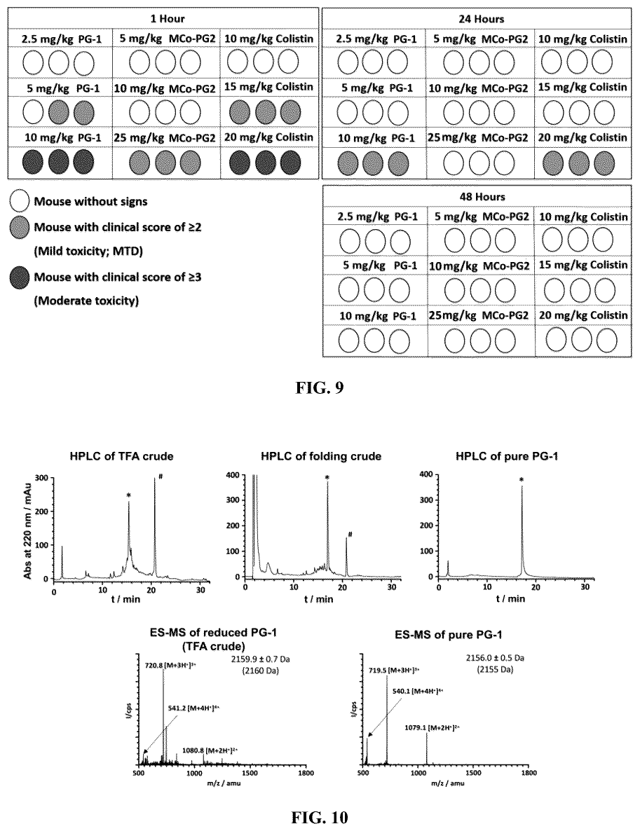

Encouraged by these results, the biological activity of cyclotide MCo-PG2 was studied in vivo. The toxicity profile of MCo-PG2 and PG-1 in Balb/c mice (n=3) was determined using intraperitoneal (i.p.) administration (FIG. 9). Colistin was used as a control antibiotic.[28] The studies revealed that intraperitoneal doses of 5 mg/kg for PG-1, 25 mg/kg for MCo-PG2 and 15 mg/kg for colistin were well tolerated by mice causing only very mild toxicity after 1 h of dosing with all recovering after 24 h (FIG. 9). This maximum tolerated dose found for colistin is consistent with previously published data.[28] Based on these results, Applicant used the corresponding compound MTDs to test the antimicrobial activity in vivo. For this purpose, Applicant employed a P. aeruginosa bacterial peritonitis model.[29] This animal model is a well-established acute infection model and is commonly utilized as a common preclinical screening method for new antibiotics.[30] Peritonitis in Balb/c mice (n=10) was established by intraperitoneal injection of 1.5×10⁷ colony forming units (CFU) per mouse of P. aeruginosa (Schroeter) Migula (ATCC 27853). The animals were then immediately treated by intraperitoneal injection with PBS, PG-1 (5 mg/kg), MCo-PG2 (10 or 25 mg/kg) and colistin (15 mg/kg). As shown in FIG. 4, single-dose administrations of 10 mg/kg and 25 mg/kg of cyclotide MCo-PG2 in the septic mice were associated with high survival rates (hazard ratio [HR]: 0.0875 and 0.048, respectively; p<0.001) comparable to those obtained in animals treated with 5 mg/kg PG-1 and 15 mg/kg colistin ([HR]: 0.040; p<0.001). After day 3 post-treatment, all the animals treated with PBS or the corresponding compound that survived were completely healthy and no further dead or moribund mice were observed over the course of the seven-day experiment (FIG. 4).

In sum, this disclosure provides the design and synthesis of a novel cyclotide with broad-spectrum antimicrobial activity in vitro against different ESKAPE pathogens (P. aeruginosa, S. aureus, K. pneumoniae, and E. coli), including 20 clinical isolates for the human pathogens P. aeruginosa and S. aureus, and more importantly in vivo using a murine model of acute P. aeruginosa peritonitis. This was successfully accomplished by grafting a series of topologically modified peptides based on the porcine protegrin PG-1 sequence onto loop 6 of the cyclotide MCoTI-I. Structural studies in solution by ¹H-NMR also revealed that the new antimicrobial cyclotide adopts a native cyclotide scaffold, allowing the grafted PG-1-based sequence to assume a bioactive native conformation. This emphasizes the tolerance of this loop in the MCoTI-based cyclotide family for the molecular engraftment of long peptide sequences.[15b, 31] For example, the sequence engrafted in the bioactive cyclotide MCo-PG2 was 18 residues long containing two extra-disulfide bonds. The most active cyclotide, MCo-PG2, displayed good antimicrobial activity against different ESKAPE pathogen strains, including P. aeruginosa, S. aureus, K. pneumoniae, and E. coli (Table 1), in addition to 20 clinical strains of P. aeruginosa and S. aureus isolated from patients with cystic fibrosis (Table 2). All the S. aureus clinical isolates were methicillin-resistant (MRSA), while around 30% of the P. aeruginosa were classified as multi-drug (MDR) strains, i.e. showing antimicrobial resistance to at least three or more antimicrobial agents from different groups of antibiotics. Cyclotide MCo-PG2 showed strong activity against these clinical strains with MIC₅₀ values of 1.5 μM against P. aeruginosa (n=20) and 6.25 μM against S. aureus (n=20) indicating its potential therapeutic value (Table 2). More importantly, MCo-PG2 (25 mg/kg, 4.5 μmol/kg; 10 mg/kg, 1.8 μmol/kg) provides a similar level of protection to that of PG-1 (5 mg/kg, 2.3 μmol/kg) and colistin (15 mg/mol, 12.3 μmol/kg) when used as single dose treatment in a murine P. aeruginosa-induced bacterial peritonitis model (FIG. 4). These results reveal that although cyclotide MCo-PG2 was in general less active than protegrin PG-1 in vitro displayed a similar level of activity to that of PG-1 in vivo. Cyclotide MCo-PG2 also exhibited 14 times less hemolytic activity than PG-1, while was only about three times less cytotoxic than PG-1 to human epithelial cells. In vivo toxicity studies also revealed that cyclotide MCo-PG2 was approximately 4 times less toxic than PG-1 in mice. These results are extremely encouraging, and open the possibility to improve even more the antimicrobial activity of cyclotide MCo-PG2 in future studies. Cyclotides contain multiple loops that are amenable to variation using different molecular evolution techniques.[32] Hence, more active cyclotides could be produced by modifying adjacent loops to loop 6 in MCo-PG2, mainly loops 1, 3 and 5 (FIG. 1). It is also worth noting that cyclotide MCo-PG2 showed a remarkable resistance to biological degradation in serum, with a t_{1/2} value of ˜60 h and not showing any significant degradation for the first 24 h (FIG. 8). In contrast, protegrin PG-1 was significantly degraded (˜₅₅% degradation) after the first 24 h under the same conditions, hence revealing the superior proteolytic stability of the circular cystine-knot topology of MCo-PG2 versus the disulfide-stabilized b-hairpin structure of PG-1. Altogether, these results show that engineered cyclotides hold great promise for the development of a novel type of peptide-based broad spectrum antimicrobial agents able to efficiently target specific bacterial targets. Applicant's results demonstrate for the first time the design of an engineered cyclotide able to show potent antimicrobial activity in vitro using physiological-like conditions and more importantly in vivo using a murine P. aeruginosa-induced peritonitis animal model, thereby providing a promising lead compound for the design of novel antibiotics. Additional supporting details are described in Ganesan et al. (2021), Engineered Cyclotides with Potent Broad In Vitro and In Vivo Antimicrobial Activity, Chemistry, A European Journal, Vol. 27, Issue 49:12702-12708 (https://doi.org/10.1002/chem.20210438), incorporated herein by reference in its entirety.

TABLE 1
Minimum inhibitory concentrations (MIC) of antimicrobial peptide PG-1 and MCo-PG2
through MCo-PG5 cyclotides. Naturally occurring protegrin PG-1 and cyclotide MCoTI-I
were used as a positive and negative controls, respectively. Antimicrobial
activities were performed by broth microdilution assays using cation-
adjusted Mueller-Hinton broth (CAMHB). This growth medium
contains 128 mM NaCl supplemented with Ca[2+] and Mg[2+]
salts providing a very similar ionic strength to
that of physiological conditions.
MIC (μM)

| | P. aeruginosa | P. aeruginosa | S. aureus | S. aureus | S. aureus | S. aureus | K. pneunoniae | K. pneunoniae | E. coli | E. coli |
|---|---|---|---|---|---|---|---|---|---|---|
| Peptide | PAO1 | PA27853 | USA300^{[a]} | 25973 | BAA977^{[b]} | HH35^{[a]} | BAA1705 | K6 | DS377 | K12 |
| PG-1 | 0.2 | 0.1 | 0.4 | 0.1 | 0.2 | 0.4 | 0.4 | 0.4 | 0.03 | 0.1 |
| MCoTI-I | >200 | >200 | >200 | >200 | >200 | >200 | >200 | >200 | >200 | >200 |
| MCo-PG2 | 1.6 | 1.6 | 6.2 | 3.1 | 3.1 | 6.2 | 12.5 | 12.5 | 0.8 | 0.8 |
| Mco-PG3 | 25 | 25 | >25 | >25 | >25 | >25 | >25 | >25 | >25 | >25 |
| MCo-PG4 | 1.6 | 1.6 | 12.5 | 12.5 | 12.5 | 12.5 | 12.5 | 12.5 | 1.6 | 1.6 |
| MCo-PG5 | 3.1 | 3.1 | 12.5 | 12.5 | 12.5 | 12.5 | 6.2 | 6.2 | 1.6 | 1.6 |

^{[a]}Methicillin resistant strain
^{[b]}Clindamycin resistant strain

Table 2. Minimum inhibitory concentration (MIC) of antimicrobial peptides MCo-PG2 and PG-1 against clinical isolates of P. aeruginosa (n=20) and methicillin-resistant S. aureus (n=20) collected from patients suffering from cystic fibrosis at the Keck Medical Center, University of Southern California. Antimicrobial activities were performed as described in

TABLE 1
Colistin and vancomycin were used as positive controls
for P. aeruginosa and S. aureus, respectively.

| | MIC (μM) | | |
|---|---|---|---|
| | PG-1 | MCo-PG2 | Colistin |
| P. aeruginosa MIC₅₀ | 0.2 | 1.5 | ≤0.2 |
| P. aeruginosa MIC₉₀ | 0.8 | 3.1 | 0.4 |
| P. aeruginosa MIC_{range} | 0.05-1.5 | 0.4-12.5 | ≤0.2-0.4 |
| | PG-1 | MCo-PG2 | Vancomycin |
| S. aureus MIC₅₀ | 0.4 | 6.3 | 0.7 |
| S. aureus MIC₉₀ | 0.8 | 12.5 | 0.4 |
| S. aureus MIC_{range} | 0.2-0.8 | 3.1-12.5 | 0.5-1.4 |

By using a topologically modified sequence of protegrin PG-1, Ganesan et al report the development of novel engineered cyclotides with effective broad-spectrum antibacterial activity against several ESKAPE bacterial strains and clinical isolates. The most active antibacterial cyclotide showed little hemolytic activity and was extremely stable in serum. In addition, this cyclotide was able to provide protection in vivo in a murine P. aeruginosa-induced peritonitis model.

### Materials and Methods

Analytical HPLC was performed on a HPI 100 series instrument with 220 nm and 280 nm detection using a Vydac C18 column (5 mm, 4.6×150 mm) at a flow rate of 1 mL/min. All runs used linear gradients of 0.1% aqueous trifluoroacetic acid (TFA, solvent A) vs. 0.1% TFA, 90% acetonitrile in H2O (solvent B). UV-vis spectroscopy was carried out on an Agilent 8453 diode array spectrophotometer. Electrospray mass spectrometry (ES-MS) analysis was performed on an Applied Biosystems API 3000 triple quadrupole electrospray mass spectrometer using software Analyst 1.4.2. Calculated masses were obtained using Analyst 1.4.2. All chemicals involved in synthesis or analysis were obtained from Aldrich (Milwaukee, WI) or Novabiochem (San Diego, CA) unless otherwise indicated.

Preparation of Fmoc-Tyr(tBu)-F. Fmoc-Tyr-F was prepared using diethylaminosulfur trifluoride DAST as previously described (34) and quickly used afterwards. Briefly, DAST (160 L, 1.2 mmol) was added drop wise at 25° C. under nitrogen current to a stirred solution of Fmoc-Tyr(tBut)-OH (459.5 mg, 1 mmol) in 10 mL of dry dichloromethane (DCM), containing dry pyridine (81 μL, 1 mmol). After 20 minutes, the mixture was washed with ice-cold water (3×20 mL). The organic layer was separated and dried over anhydrous MgSO₄. The solvent was removed under reduced pressure to give the corresponding Fmoc-amino acyl fluoride as white solid that was immediately used.

Chemical synthesis of the cyclotides. All cyclotides were synthesized by solid-phase synthesis on an automatic peptide synthesizer ABI433A (Applied Biosystems) using the Fast-Fmoc chemistry with 2-(1H-benzotriazol-1-yl)-1,1,3,3-tetramethyluronium hexafluorophosphate (HBTU)/diisopropylethylamine (DIEA) activation protocol at 0.1 mmole scale on a Fmoc-Tyr(tBu)-sulfamylbutyryl AM resin. Side-chain protection compatible with Fmoc-chemistry was employed as previously described for the synthesis of peptide α-thioesters by the Fmoc-protocol, except for the N-terminal Cys residue, which was introduced as Boc-Cys(Trt)-OH. Following chain assembly, the alkylation, thiolytic cleavage and side chain deprotection were performed for individual peptides in 1 mL polypropylene columns as previously described (35). Briefly, ˜100 mg of protected peptide-resin were first alkylated two times with ICH₂CN (174 μL, 2.4 mmol; previously filtered through basic alumina) and DIEA (82 μL, 0.46 mmol) in N-methylpyrrolidone (NMP) (2.2 mL) for 24 h. The resin was then washed with NMP (3×5 mL) and DCM (3×5 mL). The alkylated peptide resin was cleaved from the resin with HSCH₂CH₂CO₂Et (200 μL, 1.8 mmol) in the presence of a catalytic amount of sodium thiophenolate (NaSPh, 3 mg, 22 μmol) in DMF:DCM (1:2 v/v, 1.2 mL) for 24 h. The resin was then dried at reduced pressure. The side-chain protecting groups were removed by treating the dried resin with trifluoroacetic acid (TFA):H2O:tri-isopropylsilane (TIS) (95:3:2 v/v, 5 mL) for 3-4 h at room temperature. The resin was filtered and the linear peptide thioester was precipitated in cold Et₂O. The crude material was dissolved in the minimal amount of H₂O:MeCN (4:1) containing 0.1% TFA and characterized by HPLC and ES-MS as the desired grafted MCoTI-I linear precursor α-thioester (FIG. 5 and Table 3). Cyclization and folding were accomplished by flash dilution of the linear α-thioester TFA crude to a final concentration of ˜25 μM into freshly degassed 0.1 mM EDTA, 1 mM reduced glutathione (GSH), 0.1 M HEPES buffer at pH 7.4 containing 25% isopropanol for 72-96 h. Folded peptides were purified by semi-preparative HPLC using a linear gradient of 22-36% solvent B over 30 min. Pure peptides were characterized by HPLC and ES-MS (FIG. 5 and Table 3).

NMR spectroscopy. NMR samples were prepared by dissolving cyclotides into 80 mM potassium phosphate pH 6.0 in 20% d₄-MeOD, 80% (v/v) 5 mM potassium phosphate buffer at pH 6.0 (v/v) to a concentration of approximately 0.5 mM. All ¹H NMR data were recorded on either Bruker Avance III 500 MHz or Bruker Avance II 700 MHz spectrometers equipped with TCI or TXI cryoprobes. Data were acquired at 298 K, and 2,2-dimethyl-2-silapentane-5-sulfonate, DSS, was used as an internal reference. The carrier frequency was centered on the water signal, and the solvent was suppressed by using WATERGATE pulse sequence (36). Two dimensional homonuclear total coherence spectroscopy, 2D ¹H-¹H TOCSY, (spin lock time 80 ms) and two dimensional homonuclear nuclear Overhauser effect spectroscopy, 2D ¹H-¹H-NOESY (mixing time 150 ms). Spectra were collected using 4096 t₂ points and 256 t₁ of 64 transients. Spectra were processed using Topspin 2.1 (Bruker). Each 2D-data set was apodized by 90[0]-shifted sinebell-squared in all dimensions, and zero filled to 4096×512 points prior to Fourier transformation. Assignments for H[a] (H—C[a]) and H′ (H—N[a]) protons of folded MCo-PG2 (Table 4) were obtained using standard procedures (37, 38).

Human serum stability. Human serum stability. Peptides were dissolved in water at 10 mg/mL concentration. 150 μg of peptides (15 μL) were mixed with 500 μL of human serum and incubated at 37° C. Samples (30 μL) were taken at various time intervals (0-120 h) and serum proteins were precipitated using 180 μL of acetonitrile containing 0.1% TFA. After centrifugation the pellet was dissolved in 8 M GdmCl and the supernatant was lyophilized and re-dissolved in 5% acetonitrile in water containing 0.1% formic acid. Both the supernatant and solubilized pellet fractions were analyzed by HPLC and LC-MS/MS. Each experiment was done in triplicate.

Hemolysis assays. Hemolytic activity of the peptides was tested against human red blood cells (h-RBC). Single donor human red blood cells were purchased from Innovative research (IWB3ALS40ML). Prior to the experiment, h-RBC were washed three times with phosphate-buffered saline (PBS) by centrifugation for 10 min at 1,000×g and resuspended in PBS. Different concentrations of the peptide solutions were then added to 50 μL of h-RBC in PBS to give a final volume of 100 μL and a final erythrocyte concentration of 4% (v/v). The plate was incubated with agitation for 1 h at 37° C. The samples were then centrifuged at 1,000×g for 10 min. Release of hemoglobin was monitored by measuring the absorbance of the supernatant at 405 nm with a UV spectrophotometer. Controls for no hemolysis (blank) and 100% hemolysis consisted of human red blood cells suspended in PBS and 0.1% Triton X-100, respectively.

Cytotoxicity Assay. Cellular toxicities against human HEK293T and A549 epithelial cells were evaluated using Resazurin (Alamarblue™, Thermo Fisher Scientific, Waltham, MA). HEK293T cells were grown in minimum essential media (MEM), while A549 cells were grown in Dulbecco's minimum essential medium (DMEM) supplemented with 10% fetal bovine serum (FBS) and maintained at 37° C. with 5% CO₂. For each cell line, 10⁴ cells were added to each well in a 96-well polystyrene plate and incubated for 16 h. Peptides were serially diluted two-fold at concentrations ranging from 100-0.2 μM in medium containing 10% FBS in polystyrene 96 well microtiter plates (Corning) for 16 h. After the incubation period, the cells were washed with PBS and treated with 200 μL/well DMEM media supplemented with 10% FBS containing the peptides at the indicated concentration for 22 h at 37° C. in 5% CO₂ and then the medium was replaced with fresh growth medium containing resazurin and incubated for another 2 hours. Cell viability was quantified spectrophotometrically and cells incubated in the absence of peptides and containing 2% Triton X-100 (Sigma) served as controls.

Antimicrobial activity of MCo-PG cyclotides. Broad-spectrum antimicrobial activity was evaluated using the broth microdilution assays against two different pathogens from four different species of bacteria (P. aeruginosa, S. aureus, K. pneumoniae, and E. coli) to determine if the cyclotides retained its potent activity. Broth microdilution assays were utilized to determine the minimum inhibitory concentrations (MICs) of our cyclotides according to the CLSI (formerly NCCLS) guidelines modifications as described below (40). The assay utilized cation-adjusted Mueller-Hinton broth (CAMHB) (Becton, Dickinson and Company, Franklin Lakes, NJ) which was prepared according to the manufacturer's instructions. Cyclotide solutions were prepared as 10× solutions in 0.01% acetic acid. Cyclotide were serially diluted two-fold at concentrations ranging from 25-0.05 μM that contained CAMHB and 0.04% bovine serum albumin (BSA) in polypropylene microtiter plates (Corning, Corning, NY). All bacteria were incubated overnight at 37° C. at 200 rpm in CAMHB and bacterial inoculum was adjusted with additional CAMHB to 0.5 McFarland standard through spectrophotometry at 600 nm. Bacteria was then further adjusted 1:100 in CAMHB and dispensed into 96-well polypropylene microtiter plates (Corning, Corning, NY) in triplicate (corresponding to 0.5-1×10[5] CFU/well) and incubated for 24 h to determine the MIC. Using the most potent cyclotide, Applicant further evaluated its potency against cystic fibrosis isolates of P. aeruginosa and S. aureus. Study strains included a total of 20 P. aeruginosa and 20 methicillin resistant S. aureus strains from patients with cystic fibrosis at the Keck Medical Center of the University of Southern California. As many clinical isolates grow at slower rates, they were incubated for 48 h total and inspected for their MIC. Colistin sulphate (Sigma-Aldrich, St Louis, MO) was used as a reference antibiotic for P. aeruginosa and E. coli, while vancomycin hydrochloride and meropenem trihydrate were used as reference antibiotics for S. aureus and K. pneumoniae. Additional susceptibility assays were conducted with various antibiotics against the clinical isolates and determined that 30% of the P. aeruginosa clinical isolates were multi-drug resistant (MDR) and 65% of them were mucoid.

Time kill assay. The cyclotide's bactericidal kinetics was determined against a laboratory strain of P. aeruginosa (PAO1) through broth microdilution using CAMHB as described previously (41). Briefly, bacteria inoculums of 1×10[5] CFU/mL were exposed to a range of MCo-PG2 cyclotide concentrations (0.25×, 1×, 4× and 16×MIC) overtime and incubated at 37° C. over time. Aliquots of the inoculum were taken following peptide exposure at 0, 0.25, 0.5, 1, 1.5, 2, 3, 4, 6 and 24 h post treatment, then serially diluted two-fold and plated onto tryptic soy agar. The plates were incubated at 37° C. for 16 hours and CFUs were counted.

Animal studies. All animal experiments were approved by the Institutional Animal Care and Use Committee (IACUC) at the University of Southern California (Protocol #20994).

Maximum tolerated dose (MTD) toxicology Studies. The MTD was determined using two different endpoints: weight loss and clinical scoring. Clinical scores were evaluated through activity, appearance and body condition, similar to previously published literature (42). The starting doses were based on prior literature except for MCo-PG2, which we set the starting dose at 1 mg/kg. Single-dose administration was escalated two-fold until any mice met the endpoint of >15% weight loss or a clinical score>2. Any dose escalation that leads to moderate toxicity (clinical score of >2) was ceased and the dose prior served as the MTD. Mice were monitored every hour for 4 h after injection on the first day. After 24 h, mice were monitored twice daily for another two days until weight and clinical scores were returned to normal. Mice that met the criteria for moribund included a >20% weight loss and clinical score of >3 and were euthanized.

P. aeruginosa-induced peritonitis murine model. Eight to 10-week-old Balb/c mice (Jackson Laboratory, Bar Harbor, ME) were housed five in a cage with standard chow and water ad libitum. To establish the model, 1.5×10⁷ CFU of log-phase P. aeruginosa ATCC 27853 was injected intraperitoneally. Mice were intraperitoneally treated immediately with either 10 or 25 mg/kg MCo-PG2, 5 mg/kg PG-1, 15 mg/kg colistin sulphate or isotonic PBS. Mice were monitored for 7 days and/or euthanized if any mice presented signs of moribundity.

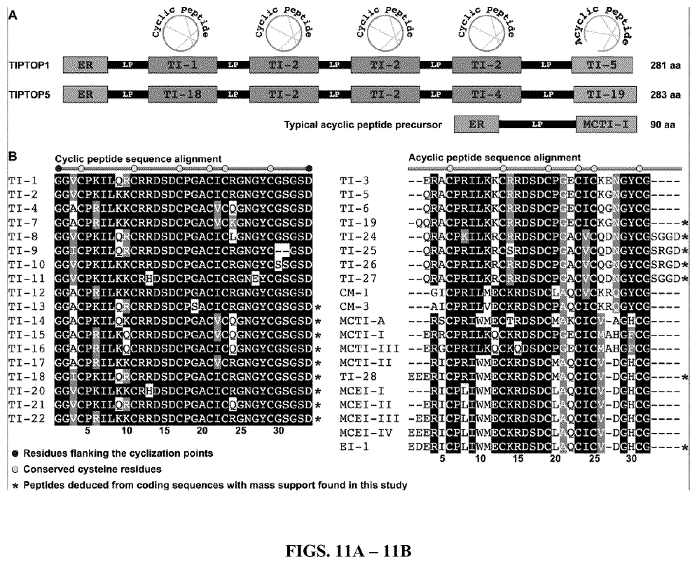

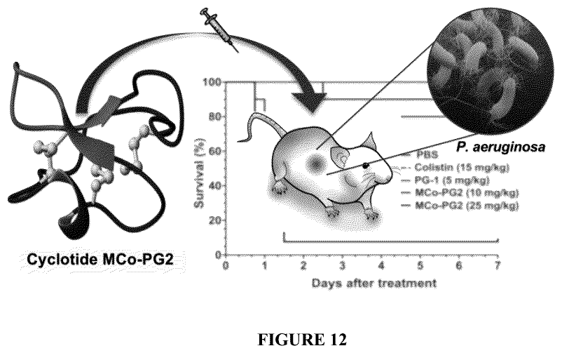

Protegrin PG-1. Protegrin PG-1 was synthesized and folded as previously described (43). Briefly, protegrin PG-1 was synthesized by solid-phase synthesis on an automatic peptide synthesizer ABI433A (Applied Biosystems) using the Fast-Fmoc chemistry with HBTU/DIEA activation protocol at 0.1 mmol scale on a Rink-amide resin. Side-chain protection compatible with Fmoc-chemistry was employed as previously described for the synthesis of peptides, Cys residues were introduced as Fmoc-Cys(Trt)-OH. Following chain assembly, side chain deprotection and resin cleavage were performed by acidolytic treatment with TFA as previously described for cyclotides (35). Briefly, side-chain protecting groups were removed by treating the dried resin with trifluoroacetic acid (TFA):H2O:tri-isopropylsilane (TIS) (95:3:2 v/v, 0.5 mL) for 3-4 h at room temperature. The resin was filtered and the linear peptide was precipitated in cold Et2O. The crude material was dissolved in the minimal amount of H2O:MeCN (4:1) containing 0.1% TFA and characterized by HPLC and ES-MS as the desired PG-1 reduced linear precursor (FIG. 10). Oxidative folding was accomplished by flash dilution of the linear PG-1 TFA crude to a final concentration of ˜25 μM into freshly degassed 0.1 mM EDTA, 1 mM oxidized glutathione, 100 mM HEPES buffer at pH 7.4 containing 25% isopropanol for 24 h. Folded PG-1 was purified by semi-preparative HPLC using a linear gradient of 18-35% solvent B over 30 min. Pure PG-1 was characterized by HPLC and ES-MS (FIG. 10) and biological activity (Tables 1, 2 and 5).

TABLE 3
Peptide sequence, molecular weight, cyclization/
folding yields for the MCo-PG
grafted cyclotides produced in this work.
Expected molecular weights are shown in parenthesis.

| Peptide | | |
|---|---|---|
| Name | Sequence | SEQ ID NO: |
| MCo-PG2 | cyclo | 293 |
| | [GGVCPKILQRCRRDSDCPGACICRGNGYC | |
| | GSGSGGRLCYCRRRFCVCVGRR] | |
| MCo-PG3 | cyclo | 294 |
| | [GGVCPKILQRCRRDSDCPGACICRGNGYC | |
| | GSGSGGCLCYCRRRFCVCVCRR] | |
| MCo-PG4 | cyclo | 295 |
| | [GGVCPKILQRCRRDSDCPGACICRGNGYC | |
| | GSGSGGGRLCYCRRRFCVCVCGRRG] | |
| MCo-PG5 | cyclo | 296 |
| | [GGVCPKILQRCRRDSDCPGACICRGNGYC | |
| | GSGSGRLCYCRRRFCVCVGR] | |

| | Molecular weight (Da) | | Cyclized/folding | |
|---|---|---|---|---|
| Peptide Name | Linear thioester | Cyclized/folded | yield (%) | time (h)^{[a]} |
| MCo-PG2 | 5636.1 ± 1.3 (5634.5) | 5504.7 ± 0.6 (5504.5) | 40 | 72 |
| MCo-PG3 | 5627.5 ± 0.4 (5627.5) | 5495.5 ± 0.4 (5495.5) | 16 | 96 |
| MCo-PG4 | 5748.1 ± 0.2 (5748.6) | 5618.6 ± 0.4 (5618.6) | 18 | 96 |
| MCo-PG5 | 5490.7 ± 1.2 (5490.3) | 5360.4 ± 0.7 (5360.3) | 20 | 96 |

^{a}Time for efficient cyclization

TABLE 4
Tabulation of chemical shifts of δ1H′ and δ1H^{α}
protons for the common residues between cyclotides MCo-PG2 and
MCoTI-I and their respective chemical shift differences.

| | δ¹H′ in | δ¹Hα in | δ¹H′ in | δ¹H^{α} in | | |
|---|---|---|---|---|---|---|
| | MCo-PG2 | MCo-PG2 | MCoTI-I | MCoTI-I | Δδ¹H′ | Δδ¹H^{α} |
| Residue^{a} | (ppm) | (ppm) | (ppm) | (ppm) | (ppm) | (ppm) |
| C1 | 8.809 | 5.426 | 8.780 | 5.391 | 0.029 | 0.035 |
| G2 | 9.905 | 4.569 | 9.902 | 4.559 | 0.003 | 0.01 |
| S3 | N/A ^{c} | N/A ^{c} | 8.715 | 4.502 | — | — |
| G4 | 9.008 | 4.238 | 9.201 | 4.403 | −0.193 | −0.165 |
| S5 | N/A ^{c} | N/A ^{c} | 8.783 | 4.542 | — | — |
| G6 | 8.289 | 4.083 | 8.237 | 4.095 | 0.052 | −0.012 |
| G7 | 8.289 | 4.083 | 8.213 | 4.015 | 0.076 | 0.068 |
| V8 | 8.425 | 4.198 | 8.434 | 4.058 | −0.009 | 0.14 |
| C9 | 8.530 | 5.338 | 8.565 | 5.122 | −0.035 | 0.216 |
| P10 | N/A ^{b} | N/A ^{b} | N/A ^{b} | N/A ^{b} | — | — |
| K11 | 8.220 | 4.330 | N/A ^{d} | N/A ^{d} | — | — |
| I12 | 7.744 | 4.460 | 7.713 | 4.425 | 0.031 | 0.035 |
| L13 | 8.798 | 4.587 | 8.693 | 4.525 | 0.105 | 0.062 |
| Q14 | 9.057 | 4.602 | N/A ^{d} | N/A ^{d} | — | — |
| R15 | 8.810 | 4.500 | 8.749 | 4.517 | 0.061 | −0.017 |
| C16 | 8.411 | 4.841 | 8.393 | 4.841 | 0.018 | 0 |
| R17 | 9.529 | 4.461 | 9.538 | 4.461 | −0.009 | 0 |
| R18 | 8.085 | 4.757 | 8.097 | 4.777 | −0.012 | −0.02 |
| D19 | N/A ^{c} | N/A ^{c} | N/A ^{d} | N/A ^{d} | — | — |
| S20 | 8.146 | 4.280 | 8.150 | 4.331 | −0.004 | −0.051 |
| D21 | 7.745 | 4.625 | 7.776 | 4.625 | −0.031 | 0 |
| C22 | 8.113 | 4.992 | 8.065 | 4.990 | 0.048 | 0.002 |
| P23 | N/A ^{b} | N/A ^{b} | N/A ^{b} | N/A ^{b} | — | — |
| G24 | 8.540 | 3.831 | 8.520 | 3.809 | 0.02 | 0.022 |
| A25 | 8.287 | 4.463 | 8.496 | 4.474 | −0.209 | −0.011 |
| C26 | 8.295 | 4.653 | 8.201 | 4.682 | 0.094 | −0.029 |
| I27 | 9.059 | 4.437 | 8.998 | 4.450 | 0.061 | −0.013 |
| C28 | 9.468 | 4.968 | 9.486 | 4.992 | −0.018 | −0.024 |
| R29 | 8.157 | 4.350 | 8.150 | 4.331 | 0.007 | 0.019 |
| G30 | N/A ^{c} | N/A ^{c} | N/A ^{d} | N/A ^{d} | — | — |
| N31 | 7.814 | 4.724 | 7.814 | 4.724 | 0 | 0 |
| G32 | 8.478 | 4.027 | 8.496 | 4.023 | −0.018 | 0.004 |
| Y33 | 7.358 | 5.268 | 7.345 | 5.297 | 0.013 | −0.029 |

^{a}Sequence numbers are based on FIG. 1.
^{b} Not available. P10 and P23 do not have amide protons.
^{c-d} H′/H^{a} cross peaks were broadened beyond detection for the following residues: S3, S5, D19, G30 in MCo-PG2 (c) and K11, Q14, D19, G30 in McoTI-I (d).

TABLE 5
Minimum inhibitory concentration (MIC) of antimicrobial peptides MCo-PG2
and PG-1 against clinical isolates of P. aeruginosa (n = 20) and methicillin-resistant
S. aureus (MRSA) (n = 20) collected from patients suffering from cystic fibrosis at
the Keck Medical Center, University of Southern California. Colistin and vancomycin
were used as positive controls for P. aeruginosa and S. aureus, respectively.

| P. aeruginosa (PA) MIC/μM | | | S. aureus (SA) MIC/μM | | |
|---|---|---|---|---|---|
| PA strain | MCo-PG2 | PG-1 | SA strain | MCo-PG2 | PG-1 |
| 466035 | 0.4 | 0.1 | 30138-2 | 3.125 | 0.4 |
| 200174 | 0.4 | 0.1 | 38878-2 | 3.125 | 0.78 |
| 167482 | 0.4 | 0.05 | 10630-1 | 6.25 | 0.2 |
| 486442 | 0.78 | 0.1 | 30493-1 | 6.25 | 0.4 |
| 618154 | 0.78 | 0.1 | 32120-1 | 6.25 | 0.4 |
| 815159 | 0.78 | 0.1 | 766 | 6.25 | 0.4 |
| 844265 | 0.78 | 0.1 | 20021-1 | 6.25 | 0.4 |
| 298473 | 0.78 | 0.2 | 87180 | 6.25 | 0.4 |
| 894496 | 0.78 | 0.2 | 10337-1 | 6.25 | 0.4 |
| 262309 | 1.5 | 0.4 | 28310 | 6.25 | 0.4 |
| 254831 | 1.5 | 0.2 | 28283 | 6.25 | 0.4 |
| 95302 | 1.57 | 0.1 | 87066 | 12.5 | 0.4 |
| 894925 | 3.125 | 0.4 | 902674 | 12.5 | 0.4 |
| 119133 | 3.125 | 0.4 | 28355 | 6.25 | 0.4 |
| 678449 | 3.125 | 1.5 | 87112 | 12.5 | 0.4 |
| 864684 | 3.125 | 0.2 | 20457-1 | 12.5 | 0.4 |
| 219686 | 3.125 | 0.4 | 4084-1 | 12.5 | 0.78 |
| 900719 | 3.125 | 0.4 | 4094 | 12.5 | 0.78 |
| 857950 | 6.25 | 0.78 | 88,531 | 12.5 | 0.78 |
| 774248 | 12.5 | 0.78 | 6151-1 | 12.5 | 0.78 |

### EQUIVALENTS

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles and concepts of the disclosure, further the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

All references cited herein are incorporated into the present disclosure to more fully describe the state of the art.

### REFERENCES

1) J. M. Pogue, K. S. Kaye, D. A. Cohen, D. Marchaim, Clin. Microbiol. Infect. 2015, 21, 302-312.
2) a) S. Santajit, N. Indrawattana, Biomed. Res. Int. 2016, 2016, 2475067; b) R. Tommasi, D. G. Brown, G. K. Walkup, J. I. Manchester, A. A. Miller, Nat. Rev. Drug Discov. 2015, 14, 529-542; c) J. N. Pendleton, S. P. Gorman, B. F. Gilmore, Expert Rev. Anti. Infect. Ther. 2013, 11, 297-308.
3) G. Wang, B. Mishra, K. Lau, T. Lushnikova, R. Golla, X. Wang, Pharmaceuticals (Basel) 2015, 8, 123-150.
4) a) R. Sher Khan, A. Iqbal, R. Malak, K. Shehryar, S. Attia, T. Ahmed, M. Ali Khan, M. Arif, M. Mii, 3 Biotech. 2019, 9, 192; b) P. M. Silva, S. Goncalves, N. C. Santos, Front. Microbiol. 2014, 5, 97.
5) a) T. Tecle, S. Tripathi, K. L. Hartshorn, Innate Immun. 2010, 16, 151-159; b) K. A. Brogden, M. Ackermann, P. B. McCray, B. F. Tack, Int. J. Antimicrob. Agents 2003, 22, 465-478.
6) a) N. Dong, C. Wang, T. Zhang, L. Zhang, C. Xue, X. Feng, C. Bi, A. Shan, Int. J. Mol. Sci. 2019, 20; b) R. Ghiselli, A. Giacometti, O. Cirioni, F. Mocchegiani, C. Silvestri, F. Orlando, W. Kamysz, A. Licci, P. Nadolski, A. Della Vittoria, J. Lukasiak, G. Scalise, V. Saba, JPEN J. Parenter. Enteral Nutr. 2007, 31, 463-468; c) S. M. Zughaier, P. Svoboda, J. Pohl, Antibiotics (Basel) 2014, 3, 694-713; d) G. Morroni, O. Simonetti, A. Brenciani, L. Brescini, W. Kamysz, E. Kamysz, D. Neubauer, M. Caffarini, M. Orciani, E. Giovanetti, A. Offidani, A. Giacometti, O. Cirioni, Med. Microbiol. Immunol. 2019, 208, 877-883.
7) a) V. N. Kokryakov, S. S. L. Harwig, E. A. Panyutich, A. A. Shevchenko, G. M. Aleshina, O. V. Shamova, H. A. Korneva, R. I. Lehrer, FEBS Lett. 1993, 327, 231-236; b) R. L. Fahrner, T. Dieckmann, S. S. Harwig, R. I. Lehrer, D. Eisenberg, J. Feigon, Chem. Biol. 1996, 3, 543-550.
8) T. Nakamura, H. Furunaka, T. Miyata, F. Tokunaga, T. Muta, S. Iwanaga, M. Niwa, T. Takao, Y. Shimonishi, J. Biol. Chem. 1988, 263, 16709-16713.
9) C. P. Hill, J. Yee, M. E. Selsted, D. Eisenberg, Science 1991, 251, 1481-1485.
10) A. Aumelas, M. Mangoni, C. Roumestand, L. Chiche, E. Despaux, G. Grassy, B. Calas, A. Chavanieu, Eur. J. Biochem. 1996, 237, 575-583.
11) a) B. Yasin, R. I. Lehrer, S. S. Harwig, E. A. Wagar, Infect. Immun. 1996, 64, 4863-4866; b) B. Yasin, S. S. Harwig, R. I Lehrer, E. A. Wagar, Infect. Immun. 1996, 64, 709-713; c) X. D. Qu, S. S. Harwig, W. M. Shafer, R. I. Lehrer, Infect. Immun. 1997, 65, 636-639.
12) a) N. Mookherjee, M. A. Anderson, H. P. Haagsman, D. J. Davidson, Nat. Rev. Drug Discov. 2020, 19, 311-332; b) D. A. Steinberg, M. A. Hurst, C. A. Fujii, A. H. Kung, J. F. Ho, F. C. Cheng, D. J. Loury, J. C. Fiddes, Antimicrob. Agents Chemother. 1997, 41, 1738-1742.
13) a) N. Soundrarajan, S. Park, Q. L. V. Chanh, H. S. Cho, G. Raghunathan, B. Ahn, H. Song, J. H. Kim, C. Park, Sci. Rep. 2019, 9; b) I A. Edwards, A. G. Elliott, A. M. Kavanagh, J. Zuegg, M. A. Blaskovich, M. A. Cooper, ACS Infect. Dis. 2016, 2, 442-450; c) N. Ostberg, Y. Kaznessis, Peptides 2005, 26, 197-206.
14) a) Y. H. Huang, Q. Du, D. J. Craik, Toxicon 2019, 172, 33-44; b) J. Weidmann, D. J. Craik, J. Exp. Bot. 2016, 67, 4801-4812.
15) a) S. J. de Veer, M. W. Kan, D. J. Craik, Chem. Rev. 2019, 119, 12375-12421; b) D. Chaudhuri, T. Aboye, J. A. Camarero, Biochem. J. 2019, 476, 67-83; c) A. Gould, J. A. Camarero, ChemBioChem 2017, 18, 1350-1363.
16) Y. Li, T. Bi, J. A. Camarero, Adv. Bot. Res. 2015, 76, 271-303.
17) a) M. J. Campbell, J. Su, J. A. Camarero, Methods Mol. Biol. 2020, 2133, 327-341; b) K. Jagadish, J. A. Camarero, Methods Mol. Biol. 2017, 1495, 41-55.
18) O. Saether, D. J. Craik, I. D. Campbell, K. Sletten, J. Juul, D. G. Norman, Biochemistry 1995, 34, 4147-4158.
19) a) L. Cascales, S. T. Henriques, M. C. Kerr, Y. H. Huang, M. J. Sweet, N. L. Daly, D. J. Craik, J. Biol. Chem. 2011, 286, 36932-36943; b) J. Contreras, A. Y. Elnagar, S. F. Hamm-Alvarez, J. A. Camarero, J. Control. Release 2011, 155, 134-143.
20) Y. Ji, S. Majumder, M. Millard, R. Borra, T. Bi, A. Y. Elnagar, N. Neamati, A. Shekhtman, J. A. Camarero, J. Am. Chem. Soc. 2013, 135, 11623-11633.
21) D. J. Craik, J. Q. Du, Curr. Opin. Chem. Biol. 2017, 38, 8-16.
22) J. F. Hernandez, J. Gagnon, L. Chiche, T. M. Nguyen, J. P. Andrieu, A. Heitz, T. Trinh Hong, T. T. Pham, D. Le Nguyen, Biochemistry 2000, 39, 5722-5730.
23) a) S. Gunasekera, F. M. Foley, R. J. Clark, L. Sando, L. J. Fabri, D. J. Craik, N. L. Daly, J. Med. Chem. 2008, 51, 7697-7704; b) P. Thongyoo, C. Bonomelli, R. J. Leatherbarrow, E. W. Tate, J. Med. Chem. 2009, 52, 6197-6200; c) L. Y. Chan, S. Gunasekera, S. T. Henriques, N. F. Worth, S. J. Le, R. J. Clark, J. H. Campbell, D. J. Craik, N. L. Daly, Blood 2011, 118, 6709-6717; d) T. L. Aboye, H. Ha, S. Majumder, F. Christ, Z. Debyser, A. Shekhtman, N. Neamati, J. A. Camarero, J. Med. Chem. 2012, 55, 10729-10734; e) W. G. Lesniak, T. Aboye, S. Chatterjee, J. A. Camarero, S. Nimmagadda, Chem. Eur. J. 2017, 23, 14469-14475.
24) a) S. S. Puttamadappa, K. Jagadish, A. Shekhtman, J. A. Camarero, Angew Chem Int Ed Engl 2010, 49, 7030-7034; Angew. Chem. 2010, 122, 7184-7188; b) S. S. Puttamadappa, K. Jagadish, A. Shekhtman, J. A. Camarero, Angew Chem Int Ed Engl 2011, 50, 6948-6949; Angew. Chem. 2011, 123, 7082-7083.
25) a) C. T. Wong, D. K. Rowlands, C. H. Wong, T. W. Lo, G. K. Nguyen, H. Y. Li, J. P. Tam, Angew Chem Int Ed Engl 2012, 51, 5620-5624; Angew. Chem. 2012, 124, 5718-5722; b) T. Aboye, C. J. Meeks, S. Majumder, A. Shekhtman, K. Rodgers, J. A. Camarero, Molecules 2016, 21, 152; c) C. D'Souza, S. T. Henriques, C. K. Wang, O. Cheneval, L. Y. Chan, N. J. Bokil, M. J. Sweet, D. J. Craik, Biochemistry 2016, 55, 396-405.
26) J. Turner, Y. Cho, N. N. Dinh, A. J. Waring, R. I. Lehrer, Antimicrob. Agents. Chemother. 1998, 42, 2206-2214.
27) L. H. Kondejewski, M. Jelokhani-Niaraki, S. W. Farmer, B. Lix, C. M. Kay, B. D. Sykes, R. E. Hancock, R. S. Hodges, J. Biol. Chem. 1999, 274, 13181-13192.
28) J. Brunetti, C. Falciani, G. Roscia, S. Pollini, S. Bindi, S. Scali, U. C. Arrieta, V. Gomez-Vallejo, L. Quercini, E. Ibba, M. Prato, G. M. Rossolini, J. Llop, L. Bracci, A. Pini, Sci. Rep. 2016, 6, 26077.
29) Y. J. Heo, Y. R. Lee, H. H. Jung, J. Lee, G. Ko, Y. H. Cho, Antimicrob. Agents Chemother. 2009, 53, 2469-2474.
30) A. J. Lewis, C. W. Seymour, M. R. Rosengart, Surg. Infect. (Larchmt.) 2016, 17, 385-393.
31) a) C. K. Wang, D. J. Craik, Nat. Chem. Biol. 2018, 14, 417-427; b) J. A. Camarero, M. J. Campbell, Biomedicines 2019, 7.
32) a) J. A. Getz, O. Cheneval, D. J. Craik, P. S. Daugherty, ACS Chem. Biol. 2013, 8, 1147-1154; b) B. Glotzbach, M. Reinwarth, N. Weber, S. Fabritz, M. Tomaszowski, H. Fittler, A. Christmann, O. Avrutina, H. Kolmar, PLoS One 2013, 8, e76956; c) T. Aboye, Y. Kuang, N. Neamati, J. A. Camarero, ChemBioChem 2015, 16, 827-833; d) Y. Li, A. Gould, T. Aboye, T. Bi, L. Breindel, A. Shekhtman, J. A. Camarero, J. Med. Chem. 2017, 60, 1916-1927; e) T. Bi, Y. Li, A. Shekhtman, J. A. Camarero, Bioorg. Med. Chem. 2018, 26, 1212-1219.
33) M. E. Felizmenio-Quimio, N. L. Daly, D. J. Craik, J. Biol. Chem. 2001, 276, 22875-22882.
34) Aboye, T., Kuang, Y., Neamati, N., and Camarero, J. A. (2015) Rapid parallel synthesis of bioactive folded cyclotides by using a tea-bag approach, ChemBioChem 16: 827-833.
35) Contreras, J., Elnagar, A. Y., Hamm-Alvarez, S. F., and Camarero, J. A. (2011) Cellular uptake of cyclotide MCoTI-I follows multiple endocytic pathways, J. Control. Release 155: 134-143.
36) Piotto, M., Saudek, V., and Sklenar, V. (1992) Gradient-tailored excitation for single-quantum NMR spectroscopy of aqueous solutions, J. Biomol. NMR 2: 661-665.
37) Cavanagh, J., and Rance, M. (1992) Suppression of cross relaxation effects in TOCSY spectra via a modified DISI-2 mixing sequence, J. Magn. Res. 96: 670-678.
38) Wuthrich, K. (1986) NMR of Proteins and Nucleic Acids.
39) Ji, Y., Majumder, S., Millard, M., Borra, R., Bi, T., Elnagar, A. Y., Neamati, N., Shekhtman, A., and Camarero, J. A. (2013) In Vivo Activation of the p53 Tumor Suppressor Pathway by an Engineered Cyclotide, J Am Chem Soc 135: 11623-11633.
40) Steinberg, D. A., Hurst, M. A., Fujii, C. A., Kung, A. H., Ho, J. F., Cheng, F. C., Loury, D. J., and Fiddes, J. C. (1997) Protegrin-1: a broad-spectrum, rapidly microbicidal peptide with in vivo activity, Antimicrob Agents Chemother 41: 1738-1742.
41) Beringer, P. M., Bensman, T. J., Ho, H., Agnello, M., Denovel, N., Nguyen, A., Wong-Beringer, A., She, R., Tran, D. Q., Moskowitz, S. M., and Selsted, M. E. (2016) Rhesus theta-defensin-1 (RTD-1) exhibits in vitro and in vivo activity against cystic fibrosis strains of Pseudomonas aeruginosa, J. Antimicrob. Chemother. 71: 181-188.
42) Aston, W. J., Hope, D. E., Nowak, A. K., Robinson, B. W., Lake, R. A., and Lesterhuis, W. J. (2017) A systematic investigation of the maximum tolerated dose of cytotoxic chemotherapy with and without supportive care in mice, BMC Cancer 17: 684.
43) Aumelas, A., Mangoni, M., Roumestand, C., Chiche, L., Despaux, E., Grassy, G., Calas, B., and Chavanieu, A. (1996) Synthesis and solution structure of the antimicrobial peptide protegrin-1, Eur. J. Biochem. 237: 575-583.
44) Fahrner, R. L., Dieckmann, T., Harwig, S. S., Lehrer, R. I., Eisenberg, D., and Feigon, J. (1996) Solution structure of protegrin-1, a broad-spectrum antimicrobial peptide from porcine leukocytes, Chem Biol 3: 543-550.

1. An antimicrobial comprising a cyclotide backbone and a protegrin PG-1 polypeptide (PG-1).

2. The antimicrobial of claim 1, wherein the PG-1 comprises the polypeptide N-X1GRLCYCRRRFCVCVGRX2-C (SEQ ID NO: 291).

3. The antimicrobial of claim 2, wherein X1 and X2 of PG-1 are the same or different an comprise 0 to 5 amino acids selected from G, R and L.

4. The antimicrobial of claim 1, wherein the PG-1 comprises the polypeptide of the group of
(SEQ ID NO: 5)
RGGRLCYCRRRFCVCVGR;
(SEQ ID NO: 6)
GGRLCYCRRRFCVCVGRR;
(SEQ ID NO: 7)
GGCLCYCRRRFCVCVCRR;
(SEQ ID NO: 8)
GGGRLCYCRRRFCVCVGRRG;
or
(SEQ ID NO: 9)
GRLCYCRRRFCVCVGR.

5. The antimicrobial of claim 1, wherein the PG-1 comprises or consists essentially of the polypeptide: GGRLCYCRRRFVCVGRR (SEQ ID NO: 292).

6. The antimicrobial of claim 1, wherein the cyclotide backbone is selected from the group of SEQ ID NOs: 1 to 4 or 10 to 290 or the Momordica spp plants, or an equivalent of each thereof, wherein the equivalent comprises a polypeptide that maintains a cystine-knot scaffold and head-to-tail cyclization but in which hypermutation of essentially all residues is permitted with the exception of the strictly conserved cysteine that comprise the knot.

7. The antimicrobial of claim 1, wherein the cyclotide backbone is a selected from the group of SEQ ID NOs: 1 to 4, or an equivalent of each thereof, wherein the equivalent comprises a polypeptide that maintains a cystine-knot scaffold and head-to-tail cyclization but in which hypermutation of essentially all residues is permitted with the exception of the strictly conserved cysteines that comprise the knot.

8. The antimicrobial of claim 1, further comprising a label or purification marker.

9. The antimicrobial of any of claim 1, further comprising a carrier.

10. A plurality of the antimicrobial of claim 1.

11. The plurality of claim 10, wherein the amino acid sequences of the plurality are the same or different from each other.

12. The plurality of claim 11, wherein the carrier is a pharmaceutically acceptable carrier.

13. The antimicrobial of claim 9, wherein the carrier further comprises an additional antibiotic or antimicrobial.

14. The plurality of claim 12, wherein the carrier further comprises an additional antibiotic or antimicrobial.

15. An isolated polynucleotide encoding the antimicrobial of claim 1 or a complement thereof.

16. A vector or isolated host cell comprising the isolated polynucleotide of claim 15.

17. A method for producing a recombinant antimicrobial, comprising growing the isolated host cell of claim 16, under conditions to express the polynucleotide.

18. A method to inhibit the growth of microorganism comprising contacting a cell comprising the microorganism or the microorganism with the antimicrobial of claim 1.

19. A method to inhibit the growth of P. aeruginosa or S. aureus comprising contacting a cell comprising the P. aeruginosa or S. aureus or the P. aeruginosa or S. aureus with antimicrobial of claim 5.

20. A method for treating a biofilm associated disease or condition in a subject in need thereof, comprising administering to the subject an effective amount of the antimicrobial of any of claim 1, thereby treating the biofilm associated disease or condition.
